(19) United States
(12) Patent Application Publication
(10) Pub. No.: US 2025/0188530 A1
(43) Pub. Date: Jun. 12, 2025

(54) METHOD OF DETECTION OF A TARGET NUCLEIC ACID SEQUENCE IN A SINGLE REACTION VESSEL

(71) Applicant: Rarity Bioscience AB, Uppsala (SE)

(72) Inventors: Ulf LANDEGREN, Uppsala (SE); Lei CHEN, Storvreta (SE)

(73) Assignee: Rarity Bioscience AB, Uppsala (SE)

(21) Appl. No.: 18/844,489

(22) PCT Filed: Mar. 8, 2023
(86) PCT No.: PCT/EP2023/055897

§ 371 Date: Sep. 6, 2024

(30) Foreign Application Priority Data
Mar. 8, 2022 (GB) 2203182.7

(51) Int. Cl. C12Q 1/6848

(52) U.S. Cl. CPC C12Q 1/6848

(57) ABSTRACT

The present disclosure and invention concerns the field of nucleic acid detection. Particularly, the present disclosure and invention relates to a method for detecting a target nucleic acid sequence in a target nucleic acid molecule in which target sequences are amplified and circularised, and, rolling circle amplification (RCA) and padlock probes are then used in a 2-stage RCA reaction, a so-called SuperRCA (sRCA), to generate a second-generation RCA product, by means of which the target nucleic acid sequence may be detected and distinguished from other nucleic acid sequences. The method has been optimised to allow it to be performed in a single reaction vessel, and may be performed in multiplex to detect different multiple target nucleic acid sequences in one or more target nucleic acid molecules. Also provided are kits for use in the method.

### FIELD

The present disclosure and invention concerns the field of nucleic acid detection. Particularly, the present disclosure and invention relates to a method for detecting a target nucleic acid sequence in a target nucleic acid molecule in which target sequences are amplified and circularised, and, rolling circle amplification (RCA) and padlock probes are then used in a 2-stage RCA reaction, a so-called SuperRCA (sRCA), to generate a second-generation RCA product, by means of which the target nucleic acid sequence may be detected and distinguished from other nucleic acid sequences. The method has been optimised to allow it to be performed in a single reaction vessel, and may be performed in multiplex to detect different multiple target nucleic acid sequences in one or more target nucleic acid molecules. Also provided are kits for use in the method.

### BACKGROUND

The detection of target nucleic acid sequences has applications in many different fields, including notably clinically, for personalised medicine and in the diagnosis, prognosis and/or treatment of disease, such as cancer, infectious diseases and inherited or genetic disorders, as well as in research and biosecurity.

The target nucleic acid sequence may readily be detected using labelled hybridisation probes, but simple hybridisation probes have relatively high lower detection limits, and cannot readily be used to discriminate between similar nucleic acid sequences. To increase sensitivity, target nucleic acid molecules containing target sequences may typically be amplified, to increase the amount of target sequence available for detection. Any of a variety of techniques known in the art may be used for the amplification, including PCR and RCA.

PCR is of course a well-known technique for exponential amplification using two primers to produce a linear amplification product. Whilst widely used, PCR suffers the drawback that errors introduced by polymerase enzyme are amplified, and as the number of PCR cycles increases, so does the rate of polymerase errors. Whilst in many applications this can be tolerated, this is a particular problem when PCR is used for the detection of variant sequences, such as mutations, particularly those which differ in only a few nucleotides, and especially where these are low-frequency or low abundance variants.

RCA utilises a strand displacement polymerase enzyme, and requires a circular amplification template. Amplification of the circular template provides a concatenated RCA product, comprising multiple copies of a sequence complementary to that of the amplification template. Such a concatemer typically forms a ball or “blob”, which may readily be visualised and detected, and thus RCA-based assays have been adopted for the detection of nucleic acids, and indeed, more generally, as reporter systems for the detection of any target analyte. Both target nucleic acids, which may themselves be circularised directly, or probes, or reporter nucleic acids more generally may provide template nucleic acid circles for RCA.

The specificity of a nucleic acid detection method may be improved by the use of probes which require dual recognition, or two binding sites for a target nucleic acid sequence, such as a padlock probe. Padlock probes are typically linear oligonucleotides with two separate target-complementary binding regions, connected by an intervening “backbone” region. When the probe has bound (hybridised) to its target nucleic acid sequence, the ends of the probe may be ligated together to circularise the probe with single nucleotide discrimination specificity within 6 bp around the ligation site. The circularised padlock probe may then be used as the template for a RCA reaction, and the RCA product may be detected. This forms the basis of a number of detection assays in use today. Padlock probes thus provide an extra layer of specificity, since only probes correctly base-paired at the ligation site will be ligated to generate the template for the molecule which will be detected.

RCA-based assays have been described which rely on secondary amplification of the initial RCA product, to increase the amount of product which is detected, and thereby to provide amplification of the signal in the assay. These include for example hyberbranched RCA which generates many unclustered subsequent RCA products through the strand displacement activity. More recently so-called “super RCA” (sRCA) reactions have been developed which comprise 2 or more rounds of RCA amplification, wherein the product of the second RCA reaction is linked to that of the first. One version of such a sRCA method is described in WO2014/0796209. In this method, a probe capable of providing or functioning as a primer is hybridised to an initial RCA product and is used to prime the amplification of a second RCA template circle which hybridises to the “primer-probe”. The second RCA template may be generated by circularisation of a padlock probe which hybridises to the “primer-probe”. In WO 2015/071445, an alternative sRCA method termed “Padlock sRCA” is described, in which a padlock probe is used to bind directly to the initial RCA product.

In many applications, the nucleic acid sequences to be detected occur at low levels, for example in the case of rare mutations, or cell-free DNA (cfDNA) in plasma or other clinical samples, or where limited amounts of sample are available. In such cases, very sensitive methods of detection are required, and particularly methods which allow a high amplification of the target nucleic acid to be achieved. PCT/EP2021/084061 describes a sRCA-based detection method of particular use in this regard, in which padlock probes bind to the target nucleic acid sequence and undergo gap-fill extension and ligation to generate many circularised copies of the target nucleic acid sequence which serve as first RCA templates for the sRCA reaction.

Whilst the sRCA methods described above go some way towards providing improved and sensitive assays, they comprise many steps which require a degree of manual involvement. Development of these methods into a commercial assay has not yet been achieved and thus they are currently subject to human error and limited to utilisation on a small scale. Optimisation of a protocol for an automatic workflow which occurs in a single vessel faces difficulties associated with interference from reagents added in preceding steps or during the protocol which remain in the reaction mixture and cannot be washed away, for example, PCR primers and polymerases. Usually, an excess of PCR primers can be avoided by ensuring their full consumption after a high number of PCR cycles. However, this approach leads to an accumulation of polymerase errors in the amplicons which is detrimental to the accuracy of the assay.

There is a continuing need for sRCA-based nucleic acid detection methods that are suitable for automation for high throughput and reliability of results without compromising sensitivity. The present disclosure, and invention, are directed to providing such a method.

### SUMMARY

In the present method, an optimised protocol has been derived, which permits the steps of generating the sRCA product for detection to be performed within a single reaction vessel.

Linear amplicons of the target nucleic acid sequence are generated, for example by PCR, and are circularised by ligation to form the first RCA template for a sRCA reaction. In order to improve sensitivity and accuracy of the method, the number of amplification cycles is kept low, to reduce the incidence of polymerase errors. As a consequence, the resulting amplification reaction mixture comprises an excess of amplification primers relative to the amplicons, as well as the amplification polymerase. Since the method is to be performed in a single vessel, and the amplicons cannot simply be separated from the rest of the reaction mixture, a clean-up step is required before ligation of the amplicons into circles can take place, to prevent interference by the primers and polymerase with the subsequent steps of the method. It is important, in this regard, that ligation occurs under conditions which prevent interference from the amplification polymerase. As described below, this can be achieved in various ways, including by degrading the primers and/or polymerase, by diluting the reaction mixture, and/or by controlling the ligation reaction to ensure ligation occurs before any unwanted extension of the 3′ end to be ligated. In this way, extension of amplicons hybridised to the ligation template, as well as unwanted extension of the amplification (e.g. PCR) primers, can be avoided.

Subsequently, the circularised amplicons are subjected to an RCA reaction to generate a first RCA product (RCP) which contains multiple complementary copies of the target sequence. The first RCP is then probed with padlock probes specific for the target sequence (specifically for its complement), and the circularised padlock probes are subjected to a second RCA reaction to generate a second RCA product, the sRCA product, which is detected, to detect the target sequence.

As with the first ligation and RCA reactions, it is important for the padlock ligation to take place under conditions which inhibit interference from any polymerases present in the reaction mixture, and further to reduce interference from any unligated padlock probes. This may similarly be achieved by the strategies outlined above, and by temperature control of the method.

Accordingly, in a first aspect, provided herein is a method for detecting a target nucleic acid sequence in a target nucleic acid molecule, said method comprising:
(a) providing a reaction mixture comprising amplicons of the target nucleic acid sequence, the amplification polymerase, and an excess of amplification primers relative to the amplicons, wherein the amplicon is a single- or double-stranded linear molecule and comprises a single copy of the target nucleic acid sequence;
(b) reducing the excess of primers from the reaction mixture of (a);
(c) if necessary denaturing double-stranded amplicons into single strands;
(d) contacting the reaction mixture with a ligation mix comprising a ligase enzyme and performing a ligation reaction to ligate the 5′ and 3′ ends of the amplicons to circularise the amplicons, wherein the ligation reaction is performed under conditions in which the ability of the amplification polymerase to extend the hybridised 3′ end of the amplicon on the ligation template is inhibited;
(e) adding to the reaction mixture from (d) an RCA mix comprising one or more RCA reagents including at least a RCA polymerase and performing a first RCA reaction using the circularised amplicons as a first RCA template to generate a first RCA product (RCP) comprising multiple repeats of a complementary copy of the target nucleic acid sequence in the amplicon;
(f) heating to inactivate the RCA polymerase;
(g) contacting the first RCP with padlock probes specific for the target nucleic acid sequence and allowing the padlock probes to hybridise to the complementary copies (or more particularly to the target sequence complements) in the multiple repeats;
h) directly or indirectly ligating the hybridised padlock probes to circularise the hybridised padlock probes, wherein the ligation reaction is performed under conditions in which the ability of polymerase to extend the hybridised 3′ ends of the padlock probes is inhibited;
(i) removing or rendering inert unligated padlock probes in the reaction mixture of (h);
(j) performing a second RCA reaction using the circularised padlock probes as second RCA templates to generate second RCPs containing multiple repeat complementary copies of the padlock probes;
wherein steps (a) to (j) are performed in a single reaction vessel and temperature is controlled during the steps; and wherein steps (g) to (j) are optionally repeated one or more times; and
(k) detecting the second or final RCPs to detect the circularised padlock probes, and thereby the target nucleic sequence.

The second RCPs (which may also be referred to as secondary RCPs) together with the first RCP to which they are attached, form the superRCA (sRCA) product, which is the product of the assay which is detected. Analogously, if the padlock probing and RCA steps are repeated, then further generations of RCPs will be attached to the previous generation of RCP, up to the final generation of RCP (the “final RCP”), together forming the sRCA product.

In particular, in step (b) of the method, the excess of primers is reduced by enzymatic digestion of the primers and/or by dilution of the reaction mixture. More particularly, in an embodiment the enzymatic digestion of the primers is performed by contacting the reaction mixture with exonuclease, or where the primers contain uracil residues, with uracil DNA glycosylase (UDG). The dilution may be performed by adding a volume of diluent to the reaction mixture. In an embodiment, the reaction mixture is diluted at least 200×.

In a more particular embodiment, the method may comprise a preceding step of performing an amplification reaction to generate amplicons of the target nucleic acid sequence. In other words, the method may comprise the step of performing an amplification reaction to provide the reaction mixture of step (a). Alternatively expressed, step (a) may comprise performing the amplification reaction, to provide the reaction mixture.

In a further more particular embodiment, the amplification reaction is a PCR reaction.

In an embodiment, step (i) of the method may comprise removing or rendering inert unligated padlock probes in the reaction mixture of (h) by enzymatic digestion, dilution, for example, by a factor of at least 4×, or extending the 3′ end of a hairpin-forming padlock probe to form a hairpin.

Typically, a ligation template is included in the ligation reaction mix to template the ligation of the amplicon. However, this is not strictly necessary and, in an embodiment, a non-templated ligation may be performed. This uses a ligase which does not require a ligation template, such as a CircLigase™ enzyme.

The methods presented above may be used to detect a variant target nucleic acid sequence in a target nucleic acid molecule. Target nucleic acid sequences may commonly occur in variant forms, for example, allelic variants, or mutant and wild-type sequences, and it may be desirable to detect which variant is present. It will thus be seen that the target nucleic acid sequence may be one of a number of different variants of the nucleic acid sequence which may occur.

Accordingly, in an embodiment, the method is for detecting a variant target nucleic acid sequence in a target nucleic acid molecule in a sample, and steps (g) to (k) comprise:
(g) contacting the first RCP with two or more padlock probes each comprising target binding regions specific for different variants of the target nucleic acid sequence and allowing the probes to hybridise to the target sequence complements in the multiple repeats;
(h) directly or indirectly ligating the padlock probes which have hybridised to their variant target sequence complements (i.e. to the variant sequence complements which are the targets of, or which correspond to the padlock probes) to circularise the hybridised padlock probes;
(i) removing or rendering inert unligated padlock probes in the reaction mixture of (h);
(j) performing second RCA reactions using the circularised padlock probes as second RCA templates to generate second RCPs containing multiple repeat complementary copies of the circularised padlock probe;
(k) detecting the final (e.g. second) RCP to identify the circularised padlock probe, and thereby the variant target nucleic acid sequence.

In step (h), correctly hybridised padlock probes are ligated, that is those that have correctly and specifically hybridised to the complements of the variant target sequences that they are designed to detect (i.e. the padlock probes which have hybridised to their respective target sequence complements).

It will thus be understood that in step (g), the padlock probes will hybridise to their intended target (i.e. specifically), where that target sequence is present.

In such uses of the method to detect variant sequences, it is generally the case that step (b) of the method comprises reducing the excess of primers from the reaction mixture of (a) by enzymatic digestion rather than by dilution. It is further preferred also to avoid using dilution in subsequent step (i) as the means of reducing unligated padlock probes in the reaction mixture of (h).

It will thus be seen that the method may be performed in multiplex using multiple different padlock probes to detect which of a number of possible different variants is present in a sample (or the levels thereof). Advantageously, however, further multiplexing is possible. Thus, the methods presented above may be performed in multiplex to detect multiple different target nucleic acid sequences in one or more target nucleic acid molecules, including the detection of multiple variants of target nucleic acid sequences in one or more target nucleic acid molecules.

In the above methods, the padlock probe may be:
(i) a 1-part padlock probe in the form of a single circularisable oligonucleotide comprising target-binding regions at its 5′ and 3′ ends; or
(ii) a 2-part padlock probe comprising a first oligonucleotide with a first, target-complementary, binding region at its 3′ end and a second binding region at its 5′ end which is complementary to a ligation template, and a second oligonucleotide with a first, target-complementary, binding region at its 5′ end and a second binding region at its 3′ end which is complementary to the ligation template; wherein the 3′ and 5′ ends of the first and second oligonucleotides are ligated together, templated respectively by the target sequence in the first RCP, and the ligation template.

Other configurations of a 2-part padlock probe are also possible.

In a second aspect, there is provided a kit for use in detecting a target nucleic acid sequence in a target nucleic acid molecule, said kit comprising:
(i) one or more different ligation templates which comprises amplicon-binding regions which are capable of hybridising to complementary binding sites in amplicons of the target nucleic acid sequence, allowing the 3′ and 5′ ends of the amplicon to be ligated;
(ii) one or more different padlock probes which comprises target-binding regions which are specific for a target nucleic acid sequence; and optionally one of more of:
(iii) an exonuclease;
(iv) proteinase K;
(v) a dilution buffer;
(vi) a ligase;
(vii) a RCA polymerase;
(viii) a RCA primer;
(ix) dNTPs;
(x) detection oligonucleotides capable of hybridising to an RCP generated using a circularised padlock probe as a RCA template.

In an embodiment, the kit is for detecting a variant target nucleic acid sequence in a target nucleic acid molecule in a sample, and may comprise in part (ii) two or more padlock probes each comprising target binding regions specific for different variants of the target nucleic acid sequence. In an embodiment, the padlock probes each comprise a detection sequence by means of which they may be distinguished from one another (that is a detection sequence which is different from that of another padlock probe).

### DETAILED DESCRIPTION

The present method provides a high-fidelity and highly-sensitive method for detecting a specific nucleotide sequence (the terms “nucleotide sequence” and “nucleic acid sequence” are used interchangeably herein). The method is particularly useful to detect variants of a target sequence, which may be present in a sample, for example mutant sequences. Particularly, rare target sequences or sequence variants, or sequences or variants present in low abundance, or at low levels, may be detected.

Amplicons of the target nucleic acid sequence, present in target nucleic acid molecules in a sample, are generated. These are circularised by ligation, and subjected to RCA to create a RCA product comprising multiple repeated tandem complementary copies of the target sequence. These amplified target sequences are then probed with a target-sequence specific padlock probe, which, upon hybridisation to the target sequence-complementary sequences, is in turn circularised (to form a second RCA template, also referred to as a secondary RCA template) and amplified by RCA, generating a second (or secondary) RCA product, by means of which the target sequence may be detected. The generation of a RCA product provides a signal that can readily be detected, and counted. A digital counting readout can be implemented. This allows for digital detection of one reaction product for each detected target molecule. Further, the products of the second RCA reaction are large products which contain multiple (hundreds, and possibly up to the order of a thousand or so) copies of complements of the target sequence, and are of significant size. Such prominent reaction products may readily be collected, with minimal risk for mix-up with any other material in the reaction.

The present method affords a high level of amplification of the target sequence, by means of which the sensitivity of the detection method is improved, rendering it capable of detecting and identifying very rare target sequences. Thus, rare sequence variants, or sequences or variants present at low levels (“low abundance”), may be detected, or identified or discriminated. Further, the method permits accurate quantification of target nucleic acid sequences. For example, the ratios of different target nucleic acid sequences may be determined with high precision, e.g. in the context of determining copy numbers of chromosomes, where target sequences from 2 different chromosomes may be detected and compared. This may be of particular utility in detection of triosomy, for example of chromosome 21, for example in the context of non-invasive prenatal testing (NIPT).

The basic principles of the detection method are shown schematically in FIG. 1. Optimisations, which are included in the method herein, allow it to be automated. Thus, the present method has been designed to allow a 2-stage RCA reaction (i.e. a sRCA) to be performed on amplicons of the target nucleic acid sequence in a single reaction vessel, without any manual input and scalable for commercial use in robotic workflows. In the interests of maintaining a low number of polymerase errors in the amplicons, it is advantageous that a low number of amplification cycles, e.g., less than 12, 11 or 10 cycles, are performed. While this has the advantage of improving sensitivity of the assay, a consequence is that an excess of amplification primers is present in the reaction mixture. If this is not addressed, the excess primers would compete with the amplicons to bind the ligation template, reducing the yield of circularised amplicons which are required for the first RCA reaction. Further, even if no ligation template is used, whilst this may reduce interference with the primers, they may still nonetheless interfere in downstream reactions. Thus, the present method has been designed to include a clean-up step to remove, or otherwise reduce, at least the amplification primers, and optionally also the amplification polymerase.

In one embodiment, the excess primers are reduced by enzymatic digestion. In another embodiment, a volume of diluent is added to the reaction mixture to dilute the reaction mixture.

Another reagent still present in the reaction mixture from the amplification step is the amplification polymerase. This can prevent ligation of the amplicon by extending the hybridised 3′ end of the amplicon on the ligation template. In one embodiment, the polymerase may be inactivated by enzymatic digestion. In another embodiment, the hybridisation of the amplicon to the ligation template may be controlled by temperature such that the 5′ end is allowed to hybridise first, followed by the 3′ end. These solutions may also be used to prevent extension of the hybridised 3′ end of the padlock probe during the second ligation step.

After the second ligation step, there is an excess of padlock probes which, if allowed to remain, may hybridise to the newly generated second RCPs and cause multiple strand displacement or interfere with detection of the second RCPs. There is therefore a need to remove these unligated, or unhybridised padlocks, or to render them inert such that they cannot participate in unwanted hybridisation or extension reactions.

In one embodiment, the unligated padlock probe is degraded by enzymatic digestion. In another embodiment, the reaction mixture is diluted, ideally by a factor of at least 4×. In yet another embodiment, the padlock probe is provided in a so-called “hairpin-forming” format, described further below, the 3′ end of which may be extended to form a hairpin which renders the probe inert. Different embodiments may be appropriate or preferred for different applications of the method and allow for a highly efficient sRCA assay which maintains a high level of sensitivity.

At its most general, the method is for detecting a target nucleic acid sequence in a target nucleic acid molecule. The term “detecting” is used broadly herein to include any means of determining the presence of the target nucleic acid sequence in the target nucleic acid molecule. In the present method, the target nucleic acid is detected by detecting the presence or amount of the second RCA product which is generated, and can include detecting simply if it is present or not, or any form of measurement of the RCA product. It will be understood in this regard, that by detecting the second or further, or final RCP, since this is attached to the first or preceding RCP, the entire sRCA product is in effect detected. Thus, the RCA product of step (j) may be detected as the “signal” for the target nucleic acid sequence. Accordingly, detecting the second RCA product in step (k) includes determining, measuring, assessing or assaying the presence or absence or amount or location of the second RCA product in any way. The presence of a second RCA product (i.e. the confirmation of its presence or amount) is indicative or identificatory of the presence of the target nucleic acid sequence, as the successful generation of the RCA product is ultimately dependent on the presence of the target nucleic acid molecule, and more particularly of the target nucleic acid sequence therein.

Quantitative and qualitative determinations, measurements or assessments are included, including semi-quantitative. Such determinations, measurements or assessments may be relative, for example, when two or more different target nucleic acid sequences, or target molecules, in a sample are being detected, or absolute. Accordingly, in an embodiment, the method may be for quantifying or determining the amount of target nucleic acid sequence which is present. The term “quantifying” when used in the context of quantifying a target nucleic acid sequence(s) in a sample can refer to absolute or to relative quantification. Absolute quantification may be accomplished by inclusion of known concentration(s) of one or more control nucleic acid molecules and/or referencing the detected level of the target nucleic acid sequence with known control nucleic acid molecules or sequences (e.g. through generation of a standard curve). Alternatively, relative quantification can be accomplished by comparison of detected levels or amounts between two or more different target nucleic acid molecules, or different target sequences, to provide a relative quantification of each of the two or more different nucleic acid molecules or sequences, i.e., relative to each other. Thus, as noted above, ratios of target nucleic acid sequences present in a sample may be determined. Thus, copy numbers of target nucleic acid molecules, e.g. chromosomes, may be compared.

The target nucleic acid sequence is a sequence in any nucleic acid molecule that it is desired to detect or identify, or in other words, the target of the assay. It may be DNA or RNA, or a modified variant thereof. Thus, the nucleic acid may be made up of ribonucleotides and/or deoxyribonucleotides as well as synthetic nucleotides that are capable of participating in Watson-Crick type or analogous base pair interactions. Thus, the nucleic acid may be or may comprise, e.g. bi-sulphite converted DNA, LNA, PNA or any other derivative containing a non-nucleotide backbone.

Typically, the target sequence will be an analyte it is desired to detect, for example a nucleic acid present in a sample, e.g. in a cell or tissue sample or any biological sample etc. Thus, it may be a naturally occurring sequence, or a derivative or copy or amplicon thereof. However, this is not necessary, and the target sequence may instead be a reporter for an analyte of an assay. Reporter nucleic acids may be used or generated in the course of an assay for any analyte, for example, a protein or other biological molecule, or small molecule, in a sample. Thus, a reporter nucleic acid may be provided as a tag, or label, for a binding probe for an analyte, and may be detected in order to detect the analyte, for example, in an immunoassay, e.g. as in an immunoPCR or immunoRCA reaction. A reporter nucleic acid may be generated in the course of an assay, for example, by a ligation reaction in a proximity ligation assay, or an extension reaction in a proximity extension assay, or by a cleavage reaction, or such like. Such a reporter target nucleic acid may therefore be a synthetic or artificial sequence.

In an embodiment, the target nucleic acid is a DNA molecule, natural or synthetic. The target nucleic acid molecule may be coding or non-coding DNA, for example genomic DNA or a sub-fraction thereof, or may be derived from genomic DNA, e.g. a copy or amplicon thereof, or it may be cDNA or a sub-fraction thereof, or an amplicon or copy thereof etc.

In another embodiment, the target nucleic acid molecule is a target RNA molecule. It may be an RNA molecule in a pool of RNA or other nucleic acid molecules, for example, genomic nucleic acids, whether human or from any source, from a transcriptome, or any other nucleic acid (e.g. organelle nucleic acids, i.e. mitochondrial or plastid nucleic acids), whether naturally occurring or synthetic. The target RNA molecule may thus be or may be derived from coding (i.e. pre-mRNA or mRNA) or non-coding RNA sequences (such as tRNA, rRNA, snoRNA, miRNA, siRNA, snRNA, exRNA, piRNA and long ncRNA). In one embodiment, the target nucleic acid molecule is a micro RNA (miRNA). In another embodiment, the target RNA molecule is 16S RNA, for example, wherein the 16S RNA is from and identificatory of a microorganism (e.g. a pathogenic microorganism) in a sample. Alternatively, the target RNA molecule may be genomic RNA, e.g. ssRNA or dsRNA of a virus having RNA as its genetic material. Notable such viruses include Ebola, HIV, SARS, SARS-COV2, influenza, hepatitis C, West Nile fever, polio and measles. Accordingly, the target RNA molecule may be positive sense RNA, negative sense RNA, or double-stranded RNA from a viral genome, or positive-sense RNA from a retroviral RNA genome.

Where the target molecule is an RNA molecule, the method may comprise a preliminary step of generating a cDNA copy of the target RNA molecule. The cDNA molecule is then amplified.

A key feature of the method is that the steps of forming the sRCA products from the target amplicons are all performed in a single reaction vessel. By this is meant a single physical, or structural, compartment, that is a physical structure which holds a reaction mixture volume. The term “compartment” as used in this context does not include droplets or emulsions, etc. as such, but this does not preclude that droplets or emulsions are used in any of the steps of the method (i.e. within a reaction vessel in steps (a) to (j)). It will further be understood that the second (or further) RCA product may be removed from the reaction vessel for the purpose of detection in step (k), for example, to transfer it to a detection instrument, such as flow cytometer, or a microscope slide etc. The vessel may thus be any vessel suitable for performing the steps of the method. Typically, this will be a reaction tube, but it may be a vessel of any format or configuration, including for example a reaction well or part of a microfluidic circuit.

The amplification which is performed to generate the target amplicons which are subjected to the method, or in more particular words, to prepare the amplification reaction mixture of (a), may be any amplification method which produces a linear amplicon which comprises a single copy of the target nucleic acid sequence. Alternatively, the amplicons in step (a) may be defined as amplicons produced by an amplification method which uses a linear template. Thus, it will be seen that this includes known amplification methods other than RCA (which uses a circular template, and produces an amplicon (RCP) comprising multiple repeats of a complementary copy of the template). Accordingly, in the methods set out above, in step (a), the amplicons may alternatively be defined as having been produced by an amplification method other than RCA, or as not including, or being, RCA products.

Typically, the amplification method will use two or more amplification primers, typically two primers.

The amplification method can thus be any known amplification method other than RCA. This may include, for example, PCR (which term is used broadly herein to include any variant of PCR), LAMP, NASB, SMAP, SDA, helicase-dependent amplification (HDA), or recombinase polymerase amplification (RPA).

Conveniently the amplification method is PCR. In one embodiment, the PCR is a conventional PCR reaction using two primers throughout the reaction. In another embodiment, the method is an asymmetric PCR. Generally speaking, an asymmetric PCR is a 2-stage reaction, comprising one stage in which both primers are used (an exponential amplification stage) and one stage in which only one of the primers is used (a linear amplification). Typically, an asymmetric PCR may be achieved by providing a limiting concentration of one of the primers, such that it becomes depleted, or exhausted, and linear amplification proceeds only with the non-limiting primer. However, this may also be achieved by design of the primers, such that they have different annealing temperatures (Tm) and temperature control is used during the method to control which primers anneal, and hence are able to prime the amplification reaction.

Thus, in a particular embodiment, the asymmetric PCR reaction which generates amplicons of the target sequence is performed using a set of primers comprising a first primer having a first melting temperature (Tm) and a second primer having a second Tm which is at least 10° C. lower than the first Tm, and the asymmetric PCR reaction comprises, in either order:
(i) an exponential PCR phase comprising no more than 12 (e.g. no more than 11 or 10 cycles, in which primers are annealed at a first annealing temperature which allows annealing of both first and second primers; and
(ii) a linear amplification phase in which primers are annealed at a second higher annealing temperature which allows annealing only of the first primer, and only one strand is amplified, thereby preferentially accumulating single-stranded amplicons of the target nucleic acid sequence.

In an embodiment, the exponential phase PCR reaction (i) is performed first, and is followed by the linear amplification reaction (ii). In this embodiment, the asymmetric PCR reaction can be said to comprise (i) a first exponential phase and (ii) a second linear amplification phase, wherein the phases are as defined above.

In the asymmetric PCR protocol above, the two PCR primers are designed to have different annealing temperatures (Tm) and temperature control is used during the method to control which primers anneal, and hence are able to prime the amplification reaction (i.e. there is a temperature “switch” in the method). Thus, the exponential reaction is performed at a lower temperature, at which both primers anneal, and the linear amplification is performed at a higher temperature at which only the higher Tm primer anneals. An increase or decrease in temperature is used to switch between the phases, depending on which phase is performed first.

As noted above, in the method in general, it is advantageous that a limited number of cycles of amplification are performed, and thus the resulting mixture contains an excess of amplification primers, relative to the amplicons. Typically, no more than 40, 39, 35, 30, 25, or 20, e.g. no more than 18, 16, 15, 14, 13, 12, 11 or 10, cycles are performed. Advantageously, to minimise polymerase errors no more than 10 cycles are performed (i.e. 10 or fewer).

Even such a limited number of cycles will result in a very large primer excess, for example, a 1000-fold excess or more. Thus, the primer excess can be at least 1000, 2000, 5000, 7000, or 10,000 fold. In some cases, it can be much higher, in the order of tens or hundreds of thousand-fold excess. By way of example, in a representative amplification reaction, the resulting reaction mixture may contain amplicons (e.g. full length PCR products) in the order of 100 fM versus primers in the order of 500 nM.

In the case of multiplex reactions where there is more than one target molecule, or more than one target sequence, the amplification of each target may be performed separately, in parallel, i.e. individual amplification reactions may be performed, each using a single set of primers (e.g. a single PCR primer pair). In this case, the individual amplification reaction mixtures, or more particularly parts or aliquots thereof may be pooled, to provide the reaction mixture of step (a). Alternatively, the amplifications of different target sequences/molecules may be performed in multiplex in the same reaction mixture. Thus, the amplification reaction may be performed using a plurality (or multiplicity) of primer sets (or in other words a pool of primers). It will be understood in this regard, that where different variants of a target sequence are being investigated, the amplicons thereof may be generated using the same set of amplification primers (in other words, the amplicons may comprise one or more of a number of different variants, which may have been amplified using the same primer set; the primers may be designed to bind at sites which flank the variant position(s)). In the case of a multiplex amplification in the same reaction mixture, the number of sequences amplified may be 2 to 10,000 or more.

Thus, to detect target sequences in two or more different target molecules, a multiplicity of amplification primer sets (e.g. primer pairs) may be used, each specific for a different target sequence, that is having target-binding regions which are complementary to primer binding sites in the target molecule which lie within or which flank the different target sequences. It will be understood in this respect, that the flanking binding sites in different target molecules will be different for different target sequences, to allow for specific binding of the first padlock probes. Alternatively or additionally, different and separate target sequences in the same target molecule may be detected, for example, different sequences in different genes on a chromosome, again using a multiplicity of different amplification primer sets, each specific for a different target sequence. However, as noted above, in a particular embodiment, the method is useful for detecting which of a number of possible different variant sequences is present in a given target molecule, for example, whether a wild-type or mutant sequence is present, or which of a number of possible mutants, or different allelic variants, or polymorphisms etc. In such a protocol, a common primer set is used to generate the amplicons, and it is then determined which variant is present by using multiple different padlock probes, each specific for a different variant of the target sequence.

The term “multiple” or “multiplicity” as used herein means two or more, for example, 3, 4, 5, 6, 8, 9, 10, 15, 20, 25, 30, 40, 50, 60, 70, 80, 90, or 100 or more. Indeed, thousands or tens of thousands of primer sets (and subsequently ligation templates or padlock probes) may be used. The number that can be used is not restricted, and can be varied. This will depend on the purpose of the method, the nature of the sample, and the target nucleic acid sequences to be detected, number of possible variants etc. Thus, to detect wild-type and mutant variants, for example, the number of different padlocks will depend on the number of different mutants possible, and may be, for example, 2-6, 2-5, 2-4 or 2-3 different padlocks. It will be appreciated that different aspects may be combined, in order to increase the overall multiplex of the assay. For example, in a given sample, the method may be used to detect different variants of different target sequences.

Where amplicons have been generated by different primers, e.g. from different target molecules, or different target sequences amplified by different primers from the same molecule, they may have different end sequences. Different or selected end sequences may be introduced by the amplification primer. Thus, the amplification primers used to amplify different target sequences may be provided with 5′ end sequences which are non-complementary to the target sequence/molecule, and which can be used to provide a common, or universal, end sequence to all amplicons. In this way, amplicons can be obtained which all have the same end sequence. This allows the use of the same ligation template to circularise all amplicons in the reaction mixture (or generated from the same sample, for example). In certain cases, it may be possible if desired for all amplicons from a group or set to be provided with the same (i.e. common) end sequence, and for different groups or sets to have different end sequences. For example, all amplicons from a given sample may be provided with the same end sequence, but amplicons from different samples, which are pooled together, may be provided with a different end sequence, allowing the same target sequences from different samples to be distinguished. Accordingly, the end sequences may be defined as being or comprising binding sites for a ligation template.

Accordingly, in an embodiment, the amplification primers (and consequently the ligation templates) may be viewed as common primers, or as generic to a group of target sequences, or a group of variant target sequences. The amplification primers may thus have target-binding regions which are complementary to binding sites (e.g. flanking regions) in the target molecule which are common to different target sequences (that is common binding sites which flank different target sequences, or different variants of a target sequence). Alternatively put, the amplification primers may have target binding regions which are common, or generic, for different target sequences, or different variants, or common for a group of target sequences or group of variants. That is, the amplification primers may have target binding regions which are capable of hybridising to complementary binding sites in the target molecule which are common to different target sequences, or to different variants of target sequences. The padlock probe is, however, specific to the target sequence, or to different variants of a target sequence, as described further below.

It will be understood in this respect, that PCR primers bind to different strands, and that accordingly, the reverse primer will bind to the strand which is the complement of the strand to which the forward primer binds. Put another way, the second, or reverse primer, will bind to the extension product of the first primer, or to the first of two strands of a double-stranded molecule. The extension product from the second primer will contain the complement of the target sequence. Accordingly, references to target sequence as used herein may, depending on context, and as appropriate, include the complement of the target sequence, i.e. they may include sequences which are complementary to the target sequence.

In a different embodiment, the amplification primers may be specific for a particular target nucleic acid sequence. Thus, for example, primer sets may be used, each specific for a different target sequence. This may have utility for diagnostic testing, e.g. NIPT, where different sequences may be detected, for example, to detect different chromosomes (and determine their copy number, for example to detect a trisomy) or in any situation where it is desired to detect one or more specific target sequences.

To perform the amplification, the target nucleic acid is contacted with the amplification primers and other reagents necessary to perform the amplification. A sample containing the target nucleic acid molecule, or a part or fraction or aliquot thereof, may be contacted with the reagents. For example, a cell or tissue sample, or any sample containing cells, or a cell extract, may be contacted with the reagents directly. The sample may be prior-treated or processed prior to the contact. In other embodiments, the target nucleic acid may first be separated, or removed from the sample. Procedures for extracting or purifying nucleic acids, e.g. DNA, from various types of sample are well known in the art. For example, the nucleic acids may be isolated from cells, or from cell-free samples, such as plasma. It may in some cases also be desirable to fragment nucleic acid molecules. Procedures for this are known in the art, and include specific digestion, e.g. using nucleases, including restriction enzymes for example, or by non-specific means, such as shearing.

To provide the amplification reaction mixture of (a), all or a part or portion of the resulting reaction mixture(s) of amplification reaction(s) which are used to generate the amplicons may be introduced, or added, to a reaction vessel (i.e. the single reaction vessel which is used in the method). For example, a 1-5 μl volume of one or more amplification reaction mixtures may added to the vessel. However, in another embodiment, the amplification may be performed in the vessel, i.e. the reaction mixture of (a) may be generated in the vessel.

In step (b) of the methods herein, the excess of amplification primers which is present in the reaction mixture of (a) is reduced. This is the first clean-up step of the method. This may be done by enzymatic digestion and/or by dilution.

In the case of enzymatic digestion, this may be achieved by contacting the reaction mixture from (a) with a digestion enzyme capable of degrading the primers. Conveniently, this may be an enzyme with exonuclease activity, particularly an enzyme with exonuclease activity as its primary catalytic activity (as opposed to a polymerase enzyme with exonuclease activity, or part thereof having exonuclease activity). Thus, an exonuclease enzyme may be used (which term in the context herein does not include a polymerase or part thereof).

The term “contacting” is used broadly herein to include bringing the reagents in question into contact. Thus, one may be added to the other and vice versa, or they may each be introduced to each other etc. However, in the context of the single vessel procedure which is performed in steps (a) to (j), contacting conveniently involves adding the reagent in question to the reaction mixture which is present in the single vessel, or in other words, adding the reagent or reagents or reagent mix in question to the vessel. Thus, in the case of step (b), this may involve adding a reagent or reagent mix containing a digestion enzyme to the reaction mixture of (a), and in particular, to the vessel which contains the reaction mixture of (a).

The exonuclease enzyme may be any suitable exonuclease enzyme. Exonucleases for performing clean-up of undesired nucleic acids are known in the art, and include for example exonuclease I, III or lambda, or mixtures thereof. The exonuclease which is selected should have strict single-stranded specific activity, for example Exol or RecJf.

Alternative digestion systems are possible. For example, if amplification primers contain uracil, then they may conveniently be digested using uracil DNA glycosylase (UDG), another well-known digestion system.

In another embodiment, the amplicon may be generated using nucleotides which are resistant to exonuclease digestion (e.g. comprising modified bases that can inhibit exonuclease activity), and primers which are composed of nucleotides which are sensitive to exonuclease digestion (e.g. normal, or conventional nucleotides). In this way, the primer part of the amplicons may be digested, whilst sparing the newly synthesised sequences in the amplicons.

This clean-up step may further include enzymatic digestion of the amplification polymerase. However, this is not strictly necessary, as interference from the polymerase may be addressed by other means. Nonetheless, in one embodiment a clean-up reagent mixture is added to the vessel which includes, in addition to the enzyme with exonuclease activity, an enzyme with proteinase activity. This may be any suitable proteinase including Serine proteases, Cysteine proteases, Threonine proteases, Aspartic Proteases, Glutamic proteases, Metalloproteases and asparagine peptide lyases, but typically this will be proteinase K. Advantageously, a thermolabile, or non-thermophilic, proteinase is used, since this may subsequently be inactivated by heat. A heating step may thus be included, to inactivate the proteinase after it has been used, as discussed further below. The use of proteinase in this step is advantageous as the proteinase will also degrade the exonuclease. In this regard, a balance will occur, between enzymatic action of the exonuclease and its digestion by proteinase. However, it has been found that a sufficient degradation of the amplification primers may occur before the exonuclease is inactivated, and it is well within the routine skill of the person skilled in the art to find and optimise reaction conditions.

The clean-up reagents, e.g. enzymes, may be provided in any suitable buffer or diluent. Advantageously, this may be a buffer suitable for the subsequent ligation and RCA reactions. The vessel may be incubated in conditions suitable for the clean-up reactions to take place. By way of representative example, this may be incubation at 37° C. for 10 minutes, but this can of course be varied, or optimised, depending on the precise nature of the enzymes used etc., and could include leaving the reaction mixture at room temperature for a period of time.

Where the method is for detecting variants, e.g. mutations, then generally speaking it is optimal for the clean-up step (b) to be performed by enzymatic digestion of the primers. Preferably, this also includes proteinase digestion of the amplification polymerase.

However, as an alternative to enzymatic digestion, dilution of the amplification reaction mixture may be employed, to dilute the primers. This may be sufficient to reduce their interference to acceptable levels. Generally speaking, this may require a significant dilution factor, which may be determined by routine experimentation. For example, a dilution factor of at least ×200 may be employed. This may be achieved by contacting the reaction mixture with a diluent, for example, adding a diluent to the reaction mixture, or by adding a part or aliquot of the amplification reaction mixture(s) to a diluent. The diluent may conveniently be a suitable buffer, for example, a buffer suitable for subsequent steps of the method. The diluent may, in another embodiment, be a reagent mix for the ligation step. In the case of a dilution step, a smaller aliquot of the individual amplification reaction mixtures, or of a pool thereof, may be contacted with the diluent, for example a 0.1-0.2 μl aliquot.

Thus, in an embodiment, steps (a) and (b) may be combined, and may be performed by adding a part or aliquot of one or more amplification reaction mixtures to a volume of clean-up reagent mix present in the vessel. This clean-up reagent mix may be a simple diluent, e.g. buffer.

After step (b), there is a step of denaturing double-stranded amplicons into single strands. This may not be necessary if the amplicons are prepared in substantially single-stranded form, for example, by an asymmetric PCR reaction. In the case of a conventional PCR (i.e. an entirely exponential phase PCR), the amplicons are generally present in double-stranded form. In such a case, the method comprises a heating step to denature the amplicons, for example, heating to 95° C. for 1-2 minutes, or similar conditions. Such a heating step may also inactivate any proteinase included in the clean-up reaction. However, generally speaking, it has been found that it is beneficial to include a separate, more gentle, heating step, e.g. incubation at 50-60° C. for 5-20 minutes, for example, 55° C. for 10 minutes, to inactivate the proteinase if it is used, and to proceed to the denaturation and ligation steps.

In this regard, in step (d), it is convenient to add the ligation mix, heat to denature, and then reduce the temperature to perform the ligation. Thus step (c) and the contacting of step (d) may be performed simultaneously or in either order. The performance of the actual ligation reaction may be controlled (i.e. the ligation reaction may be initiated) by controlling the temperature of the reaction. This may include the annealing of the ligation template (where it is used) and/or the activity of the ligase enzyme. In other words, the ligation reaction occurs after the denaturation, where that is necessary, but the reagents for the ligation, the ligase, and in some embodiments, also the ligation template, may be added to the reaction mixture from step (b) prior to or simultaneously with the denaturation of step (c). As noted above, a ligation template is typically included, as this tends to give better results, but this is not necessary, and a non-templated ligation may alternatively be performed. This may be depend on the particular method being used, and the amplicons and ligation conditions being employed etc. In one embodiment, a ligation template is included in the ligation reaction mix.

After denaturation, the temperature is reduced to allow the amplicon to hybridise (or in other words anneal) to the ligation template, where this is used, and for the ligase enzyme to catalyse the ligation reaction. Such a temperature reduction may not be necessary if there has not been a denaturation step, and the method may instead involve increasing the temperature for the ligation step (e.g. for annealing of the ligation template and/or for ligase enzyme activity).

The ligation templates comprise sequences, or regions, of complementarity to the end sequences of the amplicons. More particularly, the ligation template comprises sequences which are complementary to the ends of one strand of an amplicon. The ends of the amplicon strand hybridise to the ligation template, thereby bringing the ends of the hybridised amplicon into juxtaposition for ligation (i.e. adjacent to one another so that they can be ligated). As noted above, a ligation template can be designed to be complementary to a particular individual amplicon (in other words an amplicon comprising a particular target sequence may have a cognate ligation template, which is designed to hybridise to it, but not to other amplicons). In other embodiments, the ligation template may hybridise to a group or sub-set of amplicons, or to all amplicons in the reaction mixture, depending on the end sequences provided to the amplicons.

In this regard, it is generally the case that all amplicons (or more particularly one strand thereof which represents a particular amplicon) present in a reaction mixture are ligated. Thus, in the case of a reaction mixture containing multiple different amplicons (representing multiple different target sequences), all the different types of amplicon which may be present, or which are to be assayed, are ligated into circles. Thus, the step of circularising the amplicons by ligation can be viewed as a step of sample preparation, or of preparing a library of molecules to be amplified and probed by the padlock probes.

The ligation is performed by a ligase enzyme. This may be any suitable ligase as known in the art. Representative ligases of interest include, but are not limited to, temperature sensitive ligases such as bacteriophage T4 DNA ligase, bacteriophage T7 ligase, and E. coli ligase, and thermostable ligases such as Taq ligase, Tth ligase, Ampligase@, Pfu ligase and 9°N™ DNA Ligase. Advantageously, a thermostable or thermophilic ligase is used, since this may be retained in active form in the reaction mixture for the subsequent ligation of padlock probes in step (h). Suitable conditions for ligation are known in the art, and any reagents that are necessary and/or desirable may be combined with the reaction mixture and maintained under conditions sufficient for ligation. It will be evident that the ligation conditions may depend on the ligase enzyme used in the methods of the invention. Thus, for example, Ampligase may be used, which allows the ligation reaction to be performed at e.g. 50-60° C. (for example, at 55-60° C., e.g. 58° C.).

For performing the ligation without a ligation template, CircLigase™ ssDNA Ligase may be used. This is a thermostable ATP-dependent ligase that catalyzes intramolecular ligation (i.e. circularization) of ssDNA templates having a 5′-phosphate and a 3′-hydroxyl group.

As noted above, the ligation reaction is performed under conditions in which the ability of the amplification polymerase to extend the 3′ end of the amplicon strand which is hybridised to the ligation template is inhibited. It will be understood that such conditions will be achieved if the amplification polymerase has been digested by a proteinase in the preceding clean-up step. However, this may also be achieved by other means, most notably by controlling the hybridisation of the amplicon to the ligation template such that the 5′ end of the amplicon is hybridised first. This allows the ligation reaction to proceed, before any significant extension of the hybridised 3′ end can take place. Thus, a 2-step hybridisation may be performed, to anneal the 5′ end of the amplicon to the ligation template at a first temperature, and then reducing the temperature to allow the 3′ end of the amplicon to anneal to the ligation template.

For example, a first annealing temperature may be selected to facilitate or optimise the annealing of the 5′ end of the amplicon. For example, an annealing temperature of 50-75° C. may be used, e.g. 53 to 60° C., or more particularly, 55 to 60° C.

The annealing temperature may be reduced for annealing of the 3′ end. Again, the appropriate conditions can be selected according to what is known in the art, and the particular reagents, e.g. enzymes used. For example, after the first annealing step, the temperature may be reduced to 28-40° C., e.g. 28-35, 30-35, 28-33, 30-33, 28-32, or 30-32° C., etc., for the second annealing step.

Suitable ligase enzymes may be selected to allow such a 2-step annealing. In this regard, thermophilic ATP-dependent ligases are available which are active in the temperature range 37-95° C. Accordingly, this allows a second/annealing temperature of about 37-45° C. to be used, which is 10° C. lower than the first annealing temperature.

The skilled person will be aware how to design the ligation template and/or the amplicon ends to be able to achieve the differential annealing of the 5′ and 3′ amplicon ends, e.g. to achieve a different Tm for the annealing of the respective ends. Thus, by appropriate design and selecting appropriate conditions, including temperature, a balance may be obtained, which allows stable hybridisation of the 5′ end, followed by hybridisation of the 3′ end. For example, the sequence at the 3′ end of the amplicon which hybridises to the ligation template, may be shorter than the 5′ end sequence. By way of representative example, the 3′ end sequence may be at least 6 nucleotides long, e.g. at least 7, or 8 nucleotides long, e.g. 6-20. 6-18, 6-15, or 6-12 nucleotides long. The 5′ end sequence may be at least 12 nucleotides long, e.g. at least 13, 14, or 15 nucleotides long, for example, 12-30, 12-25, 12-20, 12-18, or 12-15 nucleotides long.

Once the amplicons have been circularised by ligation, they are subjected to RCA in step (e). RCA reactions are well known in the art, and hence the conditions for this step may be designed or selected according to protocols and principles known and described in the literature. A strand-displacing polymerase enzyme such as Phi29 or derivatives thereof is used. Conveniently, the ligation template acts as the primer for this first RCA reaction (i.e. as the “RCA primer”). However, this is not essential, and a separate RCA primer may be used. Further, if a ligation template is not used, then a RCA primer will need to be provided.

To ensure that no unwanted polymerase-catalysed extension reactions occur, reagents required for the RCA are added to the reaction mixture after the ligation step. Thus, a RCA mix containing at least the RCA polymerase is added. It is possible for other RCA reagents, e.g. dNTPs to be added before this, but generally speaking, it is convenient to add a RCA reagent mix comprising the RCA polymerase and dNTPs. The RCA reagent mix will generally include an appropriate buffer and other components to aid the RCA reaction, as is known in the art. By way of representative example, using Phi29 polymerase, the RCA reaction may proceed at 37° C., for example, for 20-40 minutes.

After the first RCA reaction, there is a step to inactivate the RCA polymerase enzyme used, e.g. by heating, prior to the subsequent steps (e.g. prior to addition of the padlock probe, or prior to ligation thereof. Such a heat inactivation step may, for example, comprise heating at 55-65° C. for a period of time, e.g. for 5-15 minutes, for example, 10 minutes.

Thus, after this heat inactivation step, the temperature may, if desired, be reduced for the subsequent steps of contacting the first RCP with the padlock probe, and allowing it to hybridise.

The RCA reaction produces a concatemeric first RCA product (RCP) comprising multiple repeat copies of the complement of the ligated amplicon. It therefore comprises multiple repeat complementary copies of the target sequence which was present in the amplicon. In this way, the target sequence is amplified. The target sequence provides a binding site for the padlock probe. The first RCP thus provides multiple binding sites for the padlock probe. Thus, more particularly, multiple copies of a given padlock probe may hybridise to the first RCP. It will, however, be understood that not every available binding site in the first RCP needs be occupied or bound by a padlock probe—it suffices that a number or multiplicity of binding sites in the first RCP are bound by the padlock probe. More particularly, a target sequence in at least one monomer of the concatemeric first RCP is bound by a padlock probe, but more particularly, in at least 2, 4, 6, 8, 10, 12, 15, 20, 25, 30, 40, 50, 80, 100, 150, 200, 300, 400, 500, 700, 1000 or more monomers.

The method relies upon multiple probes being able to hybridise to the first RCA product. Accordingly, it will be understood that the first RCA product needs to be available for probe hybridisation. This requirement is a feature of all RCA-based detection methods, where an RCA product is detected by hybridising a probe, e.g. a detection probe, to the product, and is well understood in the art. Thus, it may be advantageous for the first RCA product to have low secondary structure. However, this feature may be compensated for by performing the method in conditions which favour hybridisation, according to principles well known in the art.

The padlock probe is specific for the target sequence and thus is used to identify or detect the target sequence, or a variant thereof. The padlock probe thus comprises target-specific binding regions which are specific for a particular target sequence or for a particular variant. The target binding regions are thus designed to be complementary to a specific sequence in the target sequence, or to distinguish or discriminate between different target sequences or variants. It will be understood in this regard, that the padlock probe binds to the complement of the target sequence as it appeared in the amplicon which is subjected to the RCA. The term “target-specific” is accordingly used broadly herein to refer to the target sequence as it appears in the target molecule and its complement, and whichever of these is actually bound by the padlock probe will depend on the precise nature of the method, and which amplicon strand is ligated etc. The design of variant-specific, or allele-specific, probes is known in the art, and thus the binding regions of the padlock probe can readily be designed to detect or identify a desired target sequence or variant. In the context of variant sequence detection, the padlock probe accordingly allows genotyping of the target sequence in the first RCP and may be referred to as a genotyping padlock probe.

As discussed above, in the case of detecting a variant target sequence, two or more padlock probes may be used, each specific for a different variant sequence, and it may be detected which of these produces a second RCP in order to detect, or identify, which variant is present.

A padlock probe may alternatively be defined as a circularisable probe. The use of padlock or circularisable probes is well known in the art, including in the context of RCA reactions. A circularisable probe comprises one or more linear oligonucleotides which may be ligated together to form a circle. Padlock probes are well known and widely used and are well-reported and described in the literature. Thus, the principles of padlock probing are well understood and the design and use of padlock probes is known and described in the art. A padlock probe is typically a linear circularisable oligonucleotide which hybridises to its target nucleic acid sequence or molecule in a manner which brings 5′ and 3′ ligatable ends of the probe into juxtaposition for ligation together, either directly or indirectly, with a gap in between. By ligating the hybridised 5′ and 3′ ends of the probe, the probe is circularized. It is understood that for circularization (ligation) to occur, the ligatable 5′ end of the padlock probe has a free 5′ phosphate group.

To allow the juxtaposition of the ends of the padlock probe for ligation, the padlock probe is designed to have the target-binding sites at or near its 5′ and 3′ ends. That is, the regions of complementarity which allow binding of the padlock probe to its target lie at or near the ends of the padlock probe.

To allow ligation, the 3′ and 5′ ends which are to be ligated (the “ligatable” 3′ and 5′ ends) are hybridised to the target sequence in the first RCP, which acts as the ligation template. The ligatable ends of a padlock probe may be brought into juxtaposition for ligation in various ways, depending on the probe design. Where the target-binding sites are located at the ends of the padlock probe, the binding of the padlock probe may bring the ends into said juxtaposition. Where the complementary binding sites in the target molecule or sequence lie directly adjacent (or contiguous) to one another, the ends of the padlock probe will hybridise directly adjacent to each other (i.e. with no gap) and may be ligated to each other directly. Thus, in this case, the ligatable ends of the probe are provided by the actual ends of the probe. However, in an alternative configuration, the padlock probe is a gap-fill padlock probe, and hence the binding sites at the ends of the padlock probe do not hybridise to adjacent binding sites, but rather to non-adjacent (non-contiguous) binding sites in the target sequence. In such an arrangement, the 5′ ligatable end of the probe is provided by the actual 5′ end of the probe. However, the ligatable 3′ end of the probe is generated by extension of the hybridised 3′ end of the probe, using the target sequence as an extension template to fill the gap between the hybridised ends of the probe. The extension reaction brings the extended 3′ end of the probe into juxtaposition for ligation. In this case, the ligatable 3′ end of the probe is thus the extended 3′ end of the probe.

In another embodiment, the 3′ end of the first padlock probe may comprise a hairpin structure which comprises the 3′ target binding region. In other words, the 3′ target binding region may be at least partially comprised within a hairpin structure at the 3′ end of the padlock probe. When the probe hybridises to the target molecule, strand displacement will cause the hairpin to open, and for the 3′ end of the probe to hybridise to the target molecule.

Padlock probes may be provided in 2 or more parts that are ligated together. This may involve the provision of an additional ligation template, for example, in the case of a 2-part probe, where each part comprises only one target-binding region, and the other end of each part hybridises to a common ligation template. In another embodiment, a 2-part padlock may take the form of a “connector” oligonucleotide with two target-binding regions at or near the 5′ and 3′ ends respectively, which hybridise to the target with a gap in between them, and a gap oligonucleotide which hybridises in the gap between the ends. The gap oligonucleotide may partially or fully fill the gap. In yet another embodiment, a 2-part padlock may comprise two linear probes which comprise a target-binding region at the 5′ end of one probe and a target-binding region at the 3′ end of the other probe. The target-binding regions of the probes hybridise to their respective target sequences on the RCA product, which template the ligation of the probes into a single linear reporter molecule. The second RCA primer then templates the ligation of the remaining 5′ and 3′ ends to circularise the probe.

In a typical embodiment, however, the padlock is provided as a single circularisable oligonucleotide, whether as a gap-fill padlock or not.

In particular embodiments, the padlock probe does not have secondary structure, and more particularly does not comprise intramolecular double-stranded regions or stem-loop structures. However, dumbbell probes, which do have secondary structure, are a particular sub-type of padlock probe. The dumbbell probe comprises two stem-loop structures, joined stem to stem, wherein one of the “loops” is not closed, but is open with free 5′ and 3′ ends available for ligation to each other. This “open loop” functions as the target-binding domain of the probe.

The closed loop functions simply as a spacer to join the end of the duplex (stem). In other words, it can be seen as a padlock probe with a region of duplex formed between complementary sequences (regions) of the padlock. The region of duplex functions as a signalling domain to which an intercalating agent can bind. Thus, the “open loop” of a dumbbell probe may comprise the target-binding regions of complementarity.

In another embodiment, as noted above, the padlock probe may comprise a hairpin structure comprising the 3′ end of the probe.

Whilst padlock probes may be designed with target-specific binding regions which are specific for a particular target sequence, it will be understood that non-specific hybridisation may occur, particularly in the case of variant target sequences which are similar in sequence (e.g. where the variants are single nucleotide variants, such as SNPs, and such like). However, the high specificity of padlock probes arises from the failure of such non-specifically hybridised padlock probes to be ligated (and hence any such non-ligated padlock probes would not be amplified by the subsequent RCA step). Thus, in the second ligation step of the method herein, the padlock probes which have hybridised to their target sequence complements are ligated (that is the padlock probes which have correctly hybridised to the complement of the target sequence they are intended to detect, or in other words, their corresponding or cognate, or respective target sequence).

The second ligation (of the padlock probe) may take place under similar conditions to the first. Generally speaking, the padlock probes may be added to the reaction mixture in a ligation mix, that is in a reagent mix comprising reagents for ligation. This may be similar or the same as the ligation mix/reagents used in the preceding ligation step. In an embodiment, this may involve adding a ligase enzyme. However, if a thermostable ligase is used for the first ligation, then a further addition of ligase may not be necessary. Clearly, a separate ligation template is not necessary in this step, since ligation is templated by the first RCP to which the padlock probe binds. The second ligation is thus a templated ligation.

As for the preceding amplicon ligation in step (d), the ligation in step (h) is performed under conditions in which the ability of polymerase to extend the hybridised 3′ end of the padlock probe is inhibited. This includes amplification polymerase from the original reaction mixture and the RCA polymerase. The RCA polymerase is inactivated in the preceding step. In particular, this is to ensure that any hybridised but unligated padlock probes do not undergo any unwanted extension of their hybridised 3′ ends. It will be understood that this requirement applies to the intended ligatable 3′ end of the padlock probe. Thus, if the ligatable 3′ end is created by a gap-fill extension reaction, then the conditions for the ligation reaction are such that the ligatable 3′ end is not further extended. The strategies for ensuring such conditions are as above. Accordingly, this may involve enzymatic degradation of any polymerase added for the gap-fill extension step after the gap-fill extension has taken place. Alternatively, the extension polymerase may be inactivated by heating. A gap-fill padlock may be useful in circumstances where it is desired to incorporate a desired sequence into the second RCA template, for example, for a further amplification. In an embodiment, this may be used for NGS colony generation, for example.

In the case of a padlock probe which does not require gap-fill extension, any amplification polymerase may have been inactivated by enzymatic digestion in the first clean-up step. If such an enzymatic digestion was not included in the first clean-up step, then analogously, unwanted extension may be avoided by controlling the hybridisation of the padlock probe as discussed above, i.e. with a 2-step hybridisation of the padlock probe, using different annealing temperatures.

After the ligation step, any unligated probes in the reaction mixture are removed or rendered inert. By rendered inert is meant that the padlock probe is not able to take part in any unwanted hybridisation or ligation reactions. In particular, that the 3′ end of the probe is not capable of hybridising to any nucleic acid present in the mixture and priming an extension reaction. As discussed above, it is particularly desired to ensure that unligated padlock probes remaining in the mixture after the ligation reaction are not able to hybridise to the second RCP when it is generated. Thus, in effect, a second clean-up step is performed at this stage. A clean-up reagent mix may be added to achieve this.

The clean-up reagent may be a digestion enzyme to degrade unreacted probes, e.g. an enzyme having exonuclease activity as discussed above. Where such an enzyme is used, it is then inactivated by heating, prior to the subsequent second RCA step (step (j)). For example, if lambda exonuclease is used, the reaction mixture may be incubated at a temperature of 30-37° C. for 10-20 minutes, followed by heat inactivation at about 70-75° C. for 10-30 minutes. This can be varied appropriately depending on the precise reagents used, and the digestion and inactivation conditions are within the routine skill of the person skilled in the art to determine and optimise.

Alternatively, or additionally, a dilution step may be performed to perform the clean-up of step (i). Thus, a volume of diluent may be added to the reaction mixtures. In particular, the reaction mixture may be diluted at least 4×. As noted above, where the method is to be used for detection of a sequence variant, it is generally preferred for the clean-up to be performed by enzymatic digestion.

Alternatively, or additionally, hairpin forming padlock probes may be used. Such a probe comprises self-complementary sequences which may hybridise to one another to form a hairpin-structure comprising the 3′ end of the probe. The stem of the hairpin may be designed to have a thermal stability that does not prevent the formation of a hybrid between the loop of the hairpin structure and the target sequence in the first RCP, nor inhibit RCA. However, the stem is sufficiently stable to prime extension, and thus convert non-ligated padlock probes to full hairpins, which are unable to hybridise to or prime extension using the second RCA product as extension template. Such padlock probes which can be inactivated are termed suicide padlock probes. Suicide padlock probes are also described in U.S. Pat. No. 6,573,051.

After the clean-up step (i), the second RCA reaction is performed using the ligated padlock probes as the second RCA template. To initiate this second RCA, a second RCA reagent mix is added to the reaction mixture, which now contains the ligated padlocks. Generally speaking, this will involve adding a RCA polymerase, which may be as discussed above, and which may be provided in a reagent mix as discussed above. Whilst all the reagents for the second RCA may be added together in one reagent mix, it may be advantageous to add them separately, or step-wise. The second RCA is primed by a separate RCA primer. This may be pre-hybridised to the padlock probe or it may be separately added to the reaction mixture, or included in the RCA reagent mix. The binding site for the RCA primer may be provided in a region of the padlock probe which is different to the target binding regions (e.g. in the backbone region of the padlock, between the target-binding ends).

In one embodiment, the RCA primer for the second RCA may be included in the clean-up reagent mix. In another embodiment, it may be added separately at the same time as the clean-up reagent mix. In another embodiment, it may be added to the reaction mixture after the exonuclease for clean-up has been inactivated. The second RCA may then be initiated by adding a RCA reagent mix comprising the RCA polymerase and dNTPs. In still another embodiment, the RCA polymerase is added in a first step, and then dNTPs and RCA primer are separately added.

Where the RCA primer is included in the clean-up mix, it may be protected from digestion by the presence of modifications which render it resistant to digestion, e.g. exonuclease-resistant modifications, including for example phosphorothioate bonds. Alternatively, an exonuclease may be used in the clean-up which degrades double-stranded DNA and single-stranded DNA with a 5′ phosphate group; padlock probes have a 5′ phosphate group and so are degraded by such an exonuclease, and the RCA primer may be provided without a 5′ phosphate group, and so may be spared from digestion by the exonuclease. An example of such an exonuclease is lambda exonuclease.

In another embodiment, the clean-up of unligated padlock probes may be performed by the RCA polymerase. Thus, Phi29 has 3′ exonuclease activity, and this may be used to digest the unligated padlocks. In such an embodiment, an addition of an RCA polymerase enzyme with 3′ exonuclease may be made, and the enzyme may be allowed to perform the digestion (i.e. the reaction mixture may be incubated). Subsequently, the RCA reaction may be performed. In this case, this may be achieved by adding a primer and nucleotides to allow the RCA reaction to proceed. Thus, in this embodiment, the RCA polymerase may be added prior to the second RCA step, for a clean-up purpose (i.e. for step (i), before the second RCA reaction is performed.

As indicated above, reagents, procedures and protocols for conducting RCA reactions are known in the art and can be employed here.

The method herein requires a first RCA step, templated by the circularised amplicon, and at least a second RCA step, templated by the circularised padlock probe. The method may comprise further RCA steps, to generate a third, or further generation RCA product, using second, third, or fourth padlock probes, and so on, each targeting the target nucleic acid sequence. In other words, if desired, steps (g) to (j) may be repeated. The final generation RCA product may be detected.

It will be understood that such further generations of RCA may act to increase the signal which is ultimately detected. This may accordingly result in increased signal amplification. However, it will be also be understood that where the first and further padlock probes are gap-fill padlocks, this will result in the increased synthesis of copies of the target nucleotide sequence, or of a part thereof (depending on whether successive gap-fill reactions are fully or partially overlapping). In other words, the method may result in a clonal production of large numbers of the filled-in sequence. In still other words, the filled-in sequence (i.e. the target nucleic acid sequence, or a part thereof) may be amplified. This can be useful for preparative reactions. If multiple successive (i.e. two or more) first and further padlock probe ligation steps involve gap-fill of the same or overlapping sequences, then more copies of a specific target sequence can be generated.

The second, or as noted above, further RCP product is detected in order to detect the target nucleic acid sequence. In order to achieve this, the RCP may be rendered detectable. For example, it may be provided with a detectable label or a means by which it may be detected. Conveniently, in one embodiment, detection reagents may be used to achieve this. This is described in more detail further below. However, briefly, detection reagents for detecting the second or further RCP may be included with one or more RCA reagents added to the reaction mixture, or detection reagents may be added to the reagent mixture after the second or further RCP has been generated. Alternatively, a label may be incorporated into the RCP as it is generated, for example, using labelled nucleotides.

In one advantageous embodiment, the detection reagent is a detection oligonucleotide which is hybridised to the RCP, as described further below. The RCP is detected by detecting the detection oligonucleotide. In this regard, the detection oligonucleotide may be provided with a label, or some other detectable moiety.

To carry out the actual detection, the reaction mixture, or a part of aliquot thereof, may be removed from the vessel and moved to a detection instrument, or for further processing before detection. Thus, the contact with the detection reagents may, in an embodiment, take place outside the reaction vessel in which the preceding steps of the method were performed.

The method may be carried out in heterogenous or homogenous formats. That is, it may be performed on a solid phase (or support), or in solution or suspension (i.e. without a solid phase or support), or indeed both, since a solid phase may be introduced at a later stage, for example, at the step of detecting the second RCP, or at the stage of generating the second RCP, etc. In this regard, given that the method is performed in a single vessel, up to the point of generating the second RCP, it will be understood that any solid phase will be a solid phase that can be added to the vessel, e.g. a particulate solid phase such as beads, or the solid phase will be provided by the vessel itself (e.g. the walls and/or base of the vessel.

The format of the method may be selected based on the nature of the sample, or the target nucleic acid molecule, or the desired readout or detection technology used. In an embodiment, the method is an in-solution method, that is a method performed in a liquid phase contained in the vessel.

It will be noted from the above description that the method involves changes of temperature within and between different steps. Indeed, a feature of the method is that it is performed under temperature control. Conveniently, the method may be performed in a thermal cycling instrument. This permits a ready control of the temperature changes.

As noted above, the methods involve a number of steps in which reagents are added to the vessel containing the initial reaction mixture. The reagents are conveniently added in a buffer. In other words, the various reagent mixes used in the steps of the method discussed above typically include a buffer. Since the steps (a) to (j) are performed in the same reaction vessel without intervening washing steps, it is desirable for the buffers used for the various reagent additions to be compatible. In an embodiment, the same buffer is used for all the various reagents or reagent mixes.

The target nucleic acid molecule may be present within a sample, or obtained from a sample. The sample may be any sample which contains any amount of nucleic acid, from any source or of any origin, in which it is desired to detect a target nucleic acid sequence in a target nucleic acid molecule. A sample may thus be any clinical or non-clinical sample, and may be any biological, clinical or environmental sample in which the target nucleic acid molecule may occur.

The sample may be any sample which contains a target nucleic acid molecule, and includes both natural and synthetic samples, that is, materials which occur naturally or preparations which have been made. Naturally occurring samples may be treated or processed before being subjected to the methods herein. All biological and clinical samples are included, e.g. any cell or tissue sample of an organism, or any body fluid or preparation derived therefrom, as well as samples such as cell cultures, cell preparations, cell lysates etc. Environmental samples, e.g. soil and water samples or food samples are also included. The samples may be freshly prepared or they may be prior-treated in any convenient way, e.g. for storage.

Representative samples thus include any material which may contain a target nucleic acid molecule, including, for example, foods and allied products, clinical and environmental samples. The sample may contain any viral or cellular material, including all prokaryotic or eukaryotic cells, viruses, bacteriophages, mycoplasmas, protoplasts and organelles. Such biological material may thus comprise all types of mammalian and non-mammalian animal cells, plant cells, algae including blue green algae, fungi, bacteria, protozoa etc., or a virus. The cells may be, for example, human cells, avian cells, reptile cells etc., without limitation.

Representative samples thus include whole blood and blood-derived products such as plasma, serum and buffy coat, blood cells, urine, faeces, cerebrospinal fluid or any other body fluids (e.g. respiratory secretions, saliva, milk, etc.), tissues, biopsies, cell cultures, cell suspensions, conditioned media or other samples of cell culture constituents, etc. The sample may be pre-treated in any convenient or desired way to prepare for use in the method, for example, by cell lysis or purification, isolation of the nucleic acid, etc.

In one embodiment, the sample comprises microbial cells or viruses which have been isolated from a clinical sample or from a culture of a clinical sample. In such a sample, the target nucleic acid molecule may be a nucleotide sequence present in a microbial cell, e.g. a nucleotide sequence which is characteristic for, or discriminatory or identificatory of a microbial cell or virus, at any level, e.g. at type, group, class, genus, species or strain level.

In another embodiment, the sample may contain cell-free DNA. The sample may be a sample such as plasma or serum which directly contains cell-free DNA, or the cell-free DNA may be isolated.

In another embodiment, the sample may contain exosomes.

Since the target nucleic acid molecule need not itself be the target analyte of the assay, but can, for example, be a reporter molecule used or generated in the course of an assay for any desired analyte, the sample need not be a sample which naturally contains nucleic acid, or a source of nucleic acid (e.g. a cell or virus, or biological or clinical material etc.). As indicated above, the sample may be a synthetic or artificial sample. It may accordingly be a sample which has been subjected to a detection assay for an analyte in which a target nucleic has been generated, or to which a target nucleic acid molecule has been added. It may be a reaction mixture, or a reaction product, for example, the product resulting from an immunoassay to detect a target analyte, e.g. an immunoPCR, immunoRCA, or proximity assay (e.g. proximity ligation assay (PLA) or proximity extension assay (PEA).

The target analyte may be any analyte it is desired to detect. As discussed above, in embodiments, the target nucleic acid molecule of the method herein is the target analyte. In other embodiments, where the target nucleic acid molecule is a reporter, the target analyte may be any analyte it is desired to detect. The analyte may be a nucleic acid, a protein (which term includes peptides and polypeptides), or any other chemical or biological molecule or moiety, including, for example, carbohydrates, e.g. such as may occur as glycosyl groups on proteins. The target analyte may thus be a modified protein, for example, with a post-translational modification which is detected in an assay for an analyte.

In an embodiment, the target analyte may be a protein or component of a proteinaceous molecule which is detected on the surface of a cell, or vesicle, or other cellular or sub-cellular compartment. For example, extracellular vesicles, or exosomes, may be detected and distinguished by virtue of different proteins present on their surface. Prostasomes have been proposed as biomarkers for prostate cancer, and a particular or selected prostasome or other extracellular vesicle may be detected and distinguished by detecting one or more surface proteins thereon.

The padlock probes may comprise one or more further sequences which may serve to introduce a sequence into the ligated product, and thereby into the RCP (as a complementary copy). This may be, for example, a tag or detection sequence, e.g. a barcode or identificatory motif, or a binding site for a detection probe or primer. This is particularly the case for the padlock probe. Such a further sequence may be found, for example, in a portion of the backbone region of the padlock probe, that is the region between the target-binding regions. In a dumbbell probe, it may be in the duplex region of the probe. Tags such as barcodes or probe/primer binding sites may be designed with different needs/purposes, for example, to introduce a universal or common sequence to enable different ligated probes in a multiplex setting to be processed or detected together, e.g. a sample index or such like, or to introduce a binding site for a universal or common amplification primer. This would enable different ligated probes to be amplified together, e.g. in a library amplification by PCR or RCA.

In particular, the padlock probe may contain a detection sequence by which it may be detected. A complement of the detection sequence will become incorporated into the second (or further) RCP, and may be detected, for example, by the binding to it of a detection probe, or by sequencing. The detection sequence may be specific to the padlock probe, and thus to the target sequence, or sequence variant it is desired to detect. Thus, each padlock probe may have a different detection sequence. The detection sequence may be detected to detect or identify which padlock probe was amplified in the RCA, and hence which target sequence was present. Such a protocol may be applied, for instance, in the context of the method for detecting a target sequence variant, where each padlock is provided with a detection sequence specific to particular variant. The detection sequence may thus be seen as a marker or identification sequence. The term “detection sequence” as used herein includes both the detection sequence as it occurs in the padlock probe, and the complementary copy as it appears in the RCP.

Accordingly, a tag/barcode sequence, including particularly a detection sequence, may be used to “label” different padlock probes so that they, or their ligation or amplification products, may readily be distinguished from one another. Additionally, such a sequence may be used to tag different samples etc., for example, so that they may be pooled (i.e. a “sample” tag or marker). Thus, in a multiplex setting, different probes (i.e. probes for different target nucleic acid sequences or different variants) may be provided with different tag sequences (e.g. different marker or detection sequences) and/or they may be provided with the same tag sequence(s) e.g. for the introduction of a common or universal sequence. Such methods may be used in conjunction with particular detection methods, including the use of detection probes or sequencing methods such as sequencing by hybridisation, sequencing by ligation or other next generation sequencing chemistries, e.g. in the multiplexed detection of multiple target nucleic acids in a sample.

The term “hybridisation” or “hybridises” as used herein refers to the formation of a duplex between nucleotide sequences which are sufficiently complementary to form duplexes via Watson-Crick base pairing, or any analogous base-pair interactions. Two nucleotide sequences are “complementary” to one another when those molecules share base pair organization homology. Hence, a region of complementarity in a molecule or probe or sequence refers to a portion of that molecule or probe or sequence that is capable of forming a duplex. Hybridisation does not require 100% complementarity between the sequences, and hence regions of complementarity to one another do not require the sequences to be fully complementary, although this is not excluded. Thus, the regions of complementarity may contain one or more mismatches. Accordingly, “complementary”, as used herein, means “functionally complementary”, i.e. a level of complementarity sufficient to mediate a productive hybridisation, which encompasses degrees of complementarity less than 100%. The degree of mismatch tolerated can be controlled by suitable adjustment of the hybridisation conditions. Those skilled in the art of nucleic acid technology can determine duplex stability empirically considering a number of variables including, for example, the length and base pair composition of the respective molecules or probe oligonucleotides, ionic strength, and incidence of mismatched base pairs, following the guidance provided by the art. Thus, the design of appropriate probes or ligation templates or primers, and binding regions thereof, and the conditions under which they hybridise to their respective targets is well within the routine skill of the person skilled in the art.

A region of complementarity, such as, for example, to a target sequence in the binding region of a padlock probe, or between a detection sequence and a detection probe, or a RCA primer to the circularised padlock probe etc., may be at least 6 nucleotides long, to ensure specificity of binding, or more particularly at least 7, 8, 9 or 10 nucleotides long. The upper limit of length of the region is not critical, but may, for example, be up to 50, 40, 35, 30, 25, 20 or 15 nucleotides. A complementary region may thus have a length in a range between any one of the lower length limits and upper length limits set out above. In the case of a padlock probe, the length of an individual target-binding region may be in the lower ranges, so that the total length of the two binding regions when hybridised to their target is within the upper ranges. For example, an individual target binding region may be 8-15, e.g. 10-12 nucleotides, so that the total hybridised length is 16-30 nucleotides long, e.g. 20-24. It may be desirable, within the constraints of conformation of the probes, and spacing of the domains, and desired or favoured hybridisations, to minimise the total length of a padlock probe to minimise the size of the circle which is subjected to RCA, and hence to minimise the lengths of the complementary regions where possible.

The second RCP (and/or any further generation RCP) may be detected using any convenient protocol. Depending on the target sequence to be detected, the purpose of the method, and/or the specific details of the procedures employed in the method, the detection protocol employed may detect the RCP non-specifically or specifically.

For instance, the RCP may be detected directly, e.g. the concatemer may be cleaved to generate monomers which may be detected using gel electrophoresis, or more typically by hybridising labelled detection oligonucleotides (which may alternatively be referred to as detection probes) that hybridise to the detection sequence in the RCP, as discussed above. The detection oligonucleotide need not, however, be directly labelled. For example, the detection oligonucleotide may be an unlabelled probe which functions as a sandwich probe. The concept of sandwich probes is well known in the art and may be applied according to any convenient protocol. The sandwich probes can bind to the RCP but are not directly labelled themselves; instead, they comprise a sequence to which labelled secondary oligonucleotides can bind, thus forming a “sandwich” between the RCP and the labelled secondary oligonucleotide.

A RCP may also be detected using non-sequence-specific nucleic acid labelling methods, e.g. DNA binding stains or dyes, which are widely known in the literature, or by using labelled nucleotides for incorporation into the RCP. Alternatively, the RCP may be detected indirectly, e.g. the product may be amplified by PCR and the amplification products may be detected.

The RCP may be detected using any of the well-established methods for analysis of nucleic acid molecules known from the literature including liquid chromatography, electrophoresis, mass spectrometry, including CyTOF, microscopy, real-time PCR, fluorescent probes, microarray, colorimetric analysis such as ELISA, flow cytometry, mass spectrometry, or by turbidometric, magnetic, particle counting, electric, surface sensing, weight-based detection techniques. Generally speaking, such techniques are relevant for in solution assays.

Labelled detection oligonucleotides may be labelled with any detectable label, which may be directly or indirectly signal-giving. For example, the label may be spectroscopically or microscopically detectable, e.g. it may be a fluorescent or colorimetric label, a particle or an enzymatic label. Any of the labels used in immunohistochemical techniques may be used.

In multiplex procedures for detecting different target sequences and/or variant target sequences, different second or further RCPs may be detected and distinguished by in situ sequencing, including, for example, sequencing by synthesis, sequencing-by-hybridisation and sequencing by ligation, next generation sequencing and/or sequential barcode decoding techniques, including by sequencing-by-synthesis,-ligation or-hybridisation, and/or by using detection probes. Depending on the level of multiplexing, combinatorial labelling methods may be used, according to techniques well known in the art. For example, the large number of repeated sequences in the sRCA products can enable distinction amongst large numbers of such products via ratio labelling with fluorescent or other spectrophotometrically detectable probes. Such ratio-labelled detection probes may be used during flow cytometry, or microscopic detection techniques, e.g. imaging, to detect large numbers of sequences, e.g. the combination of at least two fluorophores at different ratios can lead to generation of multiple populations of fluorescent labels. For example, it has been found that using combinations of two fluorophores at different ratios 7 different populations can be created. This may be expanded using 3- or 4-colour combinations.

In methods which involve the use of detection oligonucleotides, the detection oligonucleotide or any secondary labelling oligonucleotide may be labelled with a directly or indirectly detectable label. A directly detectable label is one that can be directly detected without the use of additional reagents, while an indirectly detectable label is one that is detectable by employing one or more additional reagents, e.g., where the label is a member of a signal producing system made up of two or more components. In many embodiments, the label is a directly detectable label, where directly detectable labels of interest include, but are not limited to: fluorescent labels, radioisotopic labels, chemiluminescent labels, and the like. In many embodiments, the label is a fluorescent label, where the labelling reagent employed in such embodiments is a fluorescently tagged nucleotide(s), e.g. fluorescently tagged CTP (such as Cy3-CTP, Cy5-CTP) etc. Fluorescent moieties which may be used to tag nucleotides for producing labelled probe nucleic acids (i.e. detection probes) include, but are not limited to: fluorescein, the cyanine dyes, such as Cy3, Cy5, Alexa 555, Bodipy 630/650, and the like. Other labels, such as those described above, may also be employed as are known in the art.

Although various detection modalities may be employed, conveniently the second or further RCPs may be detected by microscopy or flow cytometry. In both cases directly or indirectly labelled detection oligonucleotides may be used, for example with fluorescent labels which may readily be detected. In particular, in a microscopy-based method, the RCPs may be detected by imaging.

The use of such detection techniques advantageously allow the second or further RCPs to be digitally recorded. Indeed, since the degree of signal amplification afforded by the present method allows the second or further RCPs to be visualised, they may be detected by a camera or any device including a camera, such as a mobile phone.

To detect second or further RCPs generated in a homogenous format, they may be captured or brought down to a solid support, or surface, to facilitate imaging, or microscopic detection more generally. A second RCP, being a second generation RCA product, is larger and heavier and hence readily amenable to bringing down to a surface by centrifugation. Thus, for example, tubes or plates may readily be spun to bring second RCPs down to the bottom of the tube or of a well for detection by microscopy, and particularly imaging.

A variant sequence to be detected in the method may comprise one or more variant bases. Thus, it may be a single nucleotide variant, e.g. a single nucleotide polymorphism (SNP) or mutation, or it may comprise two or more bases. Thus, a variant sequence may comprise a stretch of nucleotides, 2 or more bases of which may be variant. The variant bases may be contiguous or non-contiguous.

The length of the target sequence is not critical, and may vary according to circumstance, and the nature of the target molecule, the target sequence, or the variant position or locus. A target sequence may thus be, by way of representative example only, 1 to 10, e.g., 1-15, 1-12. 1-10, 1-8, 1-7 or 1-6 nucleotides long. In certain embodiments, however, a target sequence longer than a single nucleotide may be beneficial, to improve specificity, and in such embodiments, the target sequence may be from any one of 2, 3, 4, 5, or 6 to any one of 6, 7, 8, 9, 10, 12, 15 or 20 nucleotides long. Exemplary target sequences may thus be 4-10, 4-8, 4-7, 4-6, 5-10, 5-8, 5-7, or 6-8 nucleotides long, for example.

As noted above, also provided herein are kits for performing the methods. The kits may include the ligation templates and padlock probes as discussed above, optionally together with one or more reagents and/or instructions for use of the kit. Such reagents include dNTPs, and polymerase and ligase enzymes, as well as RCA primers for the first and/or second RCA reactions. Further, components may include buffers or other reaction components for one or more of the various reactions. Still further optional components may include means or reagents for detecting the second or further RCP. This may include, for example, detection oligonucleotides and any necessary secondary labelling reagents, including, for example, as discussed above. Further optional components may include a solid support and/or means for capture and/or immobilisation of a target nucleic acid molecule, or of a reaction component. Instructions may be, for example, in printed form, or on a computer-readable medium, or as a website address.

Advantages of the methods herein are discussed above. Such advantages are particularly beneficial in the context of detecting a target sequence or variant in complex samples, or where they are present in low abundance. As discussed above, the present method provides a high degree of signal amplification, rendering the method very sensitive. The method is thus particularly suited to detecting or identifying very rare sequence variants. The method can be used, for example, to find and detect tumour-derived mutant DNA sequences in patient samples, including notably cell-free DNA in plasma. The method may thus find utility in the diagnosis or monitoring of cancer, or e.g. to reveal recurrence of the cancer. The method may be used in the context of any cell-free DNA, and may also find application in prenatal testing including particularly NIPT. The technique is rapid, has minimal instrument requirements, and allows multiplex analysis of sequence variants for enhanced sensitivity.

The detection of low frequency or rare sequence variants or mutations also requires high specificity of detection, and a minimised risk of introducing artificial sequence alterations that could be mistaken for variant sequences. This is also afforded by the combination in the present method of the use of limited PCR cycles combined with RCA amplification, and the targeting of the target sequence by a padlock probe requiring dual target recognition.

In the present method, the first RCP is generated in a highly specific manner, and the generation of the second RCP at high amplification is dependent on the presence of the first RCP.

Each RCA product typically contains several hundreds of, or in some cases in the order of thousands of, complements of the RCA template circle (e.g. circularised amplicon or padlock probe) that was used to generate it. The second generation RCA product, which is obtained by RCA of the circularised padlock probes on the first RCA product, therefore can contain, for example, 1000×1000 monomer sequences, and is thus of a large size, and can have molecular weights which reach tens of gigaDaltons. Further, as noted above, further copies of monomer sequences may be generated by further generations of RCA reactions. Such reaction products can readily be detected. The dimensions of a second RCA product can reach several micrometers, and they may readily be visualised as individual products, e.g. by microscopy. Each second, or further, RCA product can be detected as a clonal product, generated from a single target nucleic acid molecule, without the need for compartmentalisation of the RCA reactions. For example, when labelled with fluorescent detection probes, prominent brightly fluorescent reaction products allow counting and distinction of individual amplification products of single molecules across wide fields of view at low magnification (e.g. 20×). The second or further RCA reaction products are sufficiently large and bright to be recorded by standard flow cytometry. This allows ready counting, and digital scoring in a matter of minutes using generally available instrumentation, thus offering excellent quantitative precision over wide dynamic ranges. As mentioned above, the repeat sequences present in the concatemeric second or further RCA product allow analysis of products labelled with distinct combinations of fluorophores, by ratio-labelling techniques, thereby allowing increased multiplexing. Because of their large size and considerable molecular weight, sRCA products may also be enriched by centrifugation in an ordinary desk-top centrifuge or similar. Indeed, lower speed centrifugation or unit gravity may suffice. The new method herein thus enables multiple advantages.

The strong signal amplification afforded by the second RCA reaction allows the ready and easy visualisation of signal, as discussed above, for example, microscopically at low magnification or on a digitally scanned image and hence may permit rapid and easy visual inspection of assay results in a clinical scenario, e.g. inspection of pathology results in routine use. Thus, the methods of the invention are particularly suited to clinical analysis procedures.

The methods can be helpful to identify rare integrated copies of viral genomes in human tissues or for otherwise detecting rare RCA products such as upon inefficient mutation detection in tissues. Another example when easy identification of a rare event may be helpful is when screening for the presence of circulating tumour cells (CTC) among a vast majority of non-CTC cells. The strong signal produced by the method allows fast and easy identification of events (detection of CTCs) at low magnification.

The methods allow the detection assay to be speeded up, which may be of value at point of care locations such as doctor's offices etc. In this regard, the second RCA can be performed in a relatively short time.

The ability to perform the method in a single reaction vessel is a significant advantage, allowing ready automation.

Furthermore, the increases in signal strength/speed may allow other means of detection beyond the conventional fluorescence-based methods, for example using turbidometric, magnetic, particle counting, electric, surface sensing, and weight-based detection techniques. For example, one individual sRCA product from a second generation RCA after a 1 hour amplification has the potential weight of several femtograms. Such a weight increase may be detected by methods and means known in the art such as cantilevers, surface plasmon methods, and microbalances e.g. quartz crystal microbalances etc. Further, as noted above, the increased size and weight of the second RCP allows it to efficiently be localised to a surface by centrifugation. Conveniently, this may be performed at 3000× in 15 minutes, in contrast to first generation RCA products, which cannot efficiently be captured by centrifugation using a bench-top centrifuge.

The present method can enable the generation of an enhanced signal which is localised to the product of the first RCA, and it also confers the ability to count individual reaction products (second RCA products) using standard flow cytometers or distributed on a planar surface, etc. for highly precise digital detection. In particular the second RCPs may be stained with chromogenic reagents such as HRP, and imaged via a smart phone camera. Thus, the method may permit an equivalent reaction to digital PCR, but with no need for emulsions or microfabricated structures, or conditions where exactly one template is present per compartment.

The prominent amplification products derived from the present method will further permit cloning of individual RCPs, since the product obtained from an individual first RCA template may be visualised. An individual second RCA product may therefore be identified and isolated. For example, with the aid of the present amplification method visualization can be achieved in low melt agarose for isolation with no need for magnification, and the product may then be isolated e.g. scoped out with a toothpick, analogously to the isolation of bacterial colonies.

The detection of rare mutations can be very important clinically for diagnosis. For example, mutations in certain genes (e.g. KRAS mutations) can be diagnostically important and may serve to identify the emergence of acquired resistance to particular therapies (e.g. anti-EGFR therapy). Much effort has focused in recent years on developing methods for detecting such mutations. The present method could provide a useful addition to such methods.

In addition to enabling the detection of point mutations present at low frequencies in DNA samples, the present method also provides a powerful means of screening DNA samples for the presence of any and all of a very large number of distinct target sequences in a manner that is not possible by PCR alone or any other current method. However, unlike in methods based on PCR alone, the use of padlock probes and RCA allows for several hundred thousands of probes to be applied in parallel with no deterioration of target selectivity.

Furthermore, the discrete nature of the sRCA products allows digital detection by production of one reaction product for each detected target sequence and collection of these prominent reaction products, with minimal risk for mix-up with any other material in the reaction. For example, a probe mix could be created for all types of bacteria or all species of insects or fungi. This could then be used to identify positive reaction products, for example, by amplifying tag sequences on the padlock probes by PCR, and hybridising the products to tag arrays or similar.

Still further, as noted above where second, and optionally further, ligation and RCA reactions are performed using gap-fill padlocks, then a preparative production of copies of the target nucleic acid sequence becomes possible.

The present sRCA method also increases the precision of genotyping by interrogation of the repeated sequences of individual RCA products rather than of individual target sequences. Thereby, occasional mistypings by padlock probes can be tolerated without resulting in erroneous results as long as they are considerably rarer than the correct results within an individual RCA product. This allows for genotyping via a majority-vote mechanism. This can also have consequences for how the padlock probe-based genotyping is done, where it may be possible to enhance sequence distinction by using conditions where neither variant is detected with 100% efficiency, as long as the ratio between correct and incorrect reactions is satisfactory, and a sufficient number of repeats are detected by the padlock probes.

The method will now be described in more detail with reference to the following Figures and non-limiting examples.

### DESCRIPTION OF FIGURES

FIG. 1: schematic illustration of the method for generation of SuperRCA amplification products. A) DNA sequences of interest in a sample are amplified by PCR. B) Amplified strands are converted to single-stranded DNA circles via templated ligation of their 5′ and 3′ ends. C) Oligonucleotides that template the circularization reactions next serve as primers for RCA reactions. D) The RCA products are then interrogated with padlock probes specific for mutant or wildtype sequences. E) Ligated padlock probes, wound around the RCA products, thereafter template secondary RCA reactions, primed by an added oligonucleotide. F) For each starting DNA circle, the reaction gives rise to large clusters of mainly single-stranded DNA objects, called SuperRCA products. Up to a million fluorescence-labelled hybridisation probes can bind each of the mutant- or wildtype-specific products, allowing efficient counting via e.g. standard flow cytometry.

FIG. 2: Flow cytometry readout data of single tube SuperRCA assays revealing the IDH2 R172K mutation at (a) 1.024%, (b) 0.064%, (c) 0.001%, and (d) wild-type samples which are negative for the IDH2 R172K mutation.

FIG. 3: Analysis of four-fold serial dilutions of genomic DNA from cells with IDH2 p.R172K point mutation using the SuperRCA procedure or ddPCR. Genomic DNA samples with mutant genomic DNA serially diluted in wildtype genomic DNA were each divided into two portions and analyzed with the SuperRCA or ddPCR assays. 330 ng DNA, corresponding to 100,000 human haploid genomes, were analyzed per sample for the SuperRCA assay, while 66 ng, or 20,000 human haploid genome equivalents, were used for ddPCR, according to the manufacturer's instructions. Each data point was calculated as means of three biological replicates, the limit of detection (LoD) for ddPCR and SuperRCA assay for each analyte was calculated respectively (LoD=Mean (Wildtype)+3×SD (wildtype)) and presented in the figures with dash lines.

FIG. 4: Patient UPN126 was diagnosed with primary myelofibrosis two years prior to AML diagnosis. The bone marrow sample at AML diagnosis (day 0) revealed high levels of the IDH2 p.R140Q mutation as recorded via NGS, SuperRCA and ddPCR. Retrospective analysis of the initial bone marrow sample in the myelofibrosis stage was negative for the IDH2 p.R140Q mutation by SuperRCA and ddPCR assays. SCT was performed on day 139, at which point considerable levels of the mutation remained. The IDH2 p.R140Q mutation was detected at low levels by the SuperRCA assay approximately three months post SCT, whereas ddPCR failed to identify the detectable levels of the mutation at that timepoint. All subsequent samples have been negative in blood and bone marrow by SuperRCA and ddPCR analysis, indicating a complete remission.

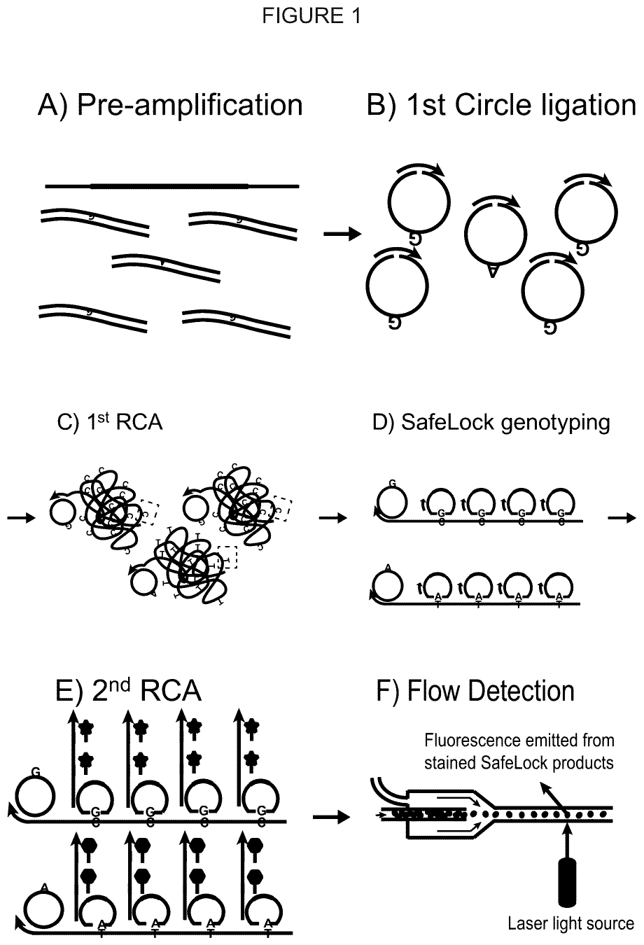

### EXAMPLES

### Materials and Methods

### Extraction of Genomic DNA.

DNA was extracted from BM cells or from whole blood using the QIAamp DNA Blood mini kit (Qiagen cat.51104) and eluted in 50 μL elution buffer.

### PCR Based Library Prep: Tube A

### High Fidelity PCR Pre-Amplification:

Sequences of interest in genomic DNA were amplified with SuperFi DNA polymerase (Thermo Scientific) in 25 μl PCR reactions containing 1λ SuperFi buffer, 0.2 mM dNTP, 100 nM Fwd/Rev PCR primers, 330 ng gDNA and 0.0005 U/μl SuperFi DNA polymerase. The PCR program was as follows: 98° C. for 30 sec, 10 cycles of 98° C. for 15 sec, 62° C. for 120 sec, and a final elongation at 72° C. for 5 min.

### SuperRCA Assay: Tube B

### Primer and PCR Polymerase Clean Up:

1 μL per target from the PCR based library prep was mixed with 20 μL clean-up solution containing 1λ SuperRCA buffer (Rarity Bioscience AB), 0.125 μL Exol (Thermo Fisher Inc.), and 0.0006 U/L Thermoliable Proteinase K. The mixture were incubated at 37° C. for 10 min, followed by 55° C. for 10 min.

### Ligase-Mediated Circularization of One Strand of the PCR Products:

20 μl ligation solution containing 1× SuperRCA buffer (Rarity Bioscience), 100 nM of ligation template, complementary to both ends of one strand of the amplification products, 0.5 mM NAD (Sigma) and 2 U Ampligase (Lucigen) were added into the clean-up solution containing amplified PCR products. The mixtures were incubated at 95° C. for 1 min, followed by 58° C. for 30 min.

### Target Sequence Amplification by a First RCA:

Circularized strands of PCR products containing target nucleotide positions were amplified by RCA. 5 μl of 1× SuperRCA buffer (Rarity Bioscience), 1.8 mM dNTP (Invitrogen), and 2.5 U Phi29 polymerase (New England Biolabs) were added to the circularized products. The reactions were incubated at 37° C. for 30 min, then at 65° C. for 10 min.

### Genotyping of RCA Products Via Padlock Probe Ligation:

Padlock probes were hybridised to first-generation RCA products and ligated in a sequence-specific manner, by adding 5 μL ligation mix containing 1× SuperRCA buffer (Rarity Bioscience), 3 mM NAD (Sigma), 2.5 U Ampligase (Lucigen), and 60 nM genotyping padlock probe pairs to the reaction mixtures, incubating at 55° C. for 30 min.

### Digestion of the Non-Reacted Genotyping Probes:

5 μL clean-up solution containing 1× SuperRCA buffer (Rarity Bioscience AB), 1.2 μM primer and 1 U/μL Lambda exo was added into the reaction mixture and incubated at 37° C. for 15 min then 75° C. for 20 min.

### Secondary RCA Templated by Padlock Probes Bound to Primary RCA Products:

5 μL RCA mixture containing 1× SuperRCA buffer (Rarity Bioscience), 0.6 mM dNTPs and 6 U Phi29 DNA polymerase (New England Biolabs) was added to the mixture and the reactions were incubated at 37° C. for 30 min.

### Digital Recording of SuperRCA Products by Flow Cytometry:

The final reaction mixtures containing SuperRCA products were diluted into hybridisation buffer containing 100 nM fluorophore-labelled oligonucleotide probes specific for the different SuperRCA products, in 1× SuperRCA buffer (Rarity Bioscience) to a final volume of 250 μL. The solutions were applied onto the CytoFlex flow cytometer (Beckman Coulter) and SuperRCA products were counted at ‘Medium’ speed (30 μl/minute) for 150 seconds per sample.

### Example 1

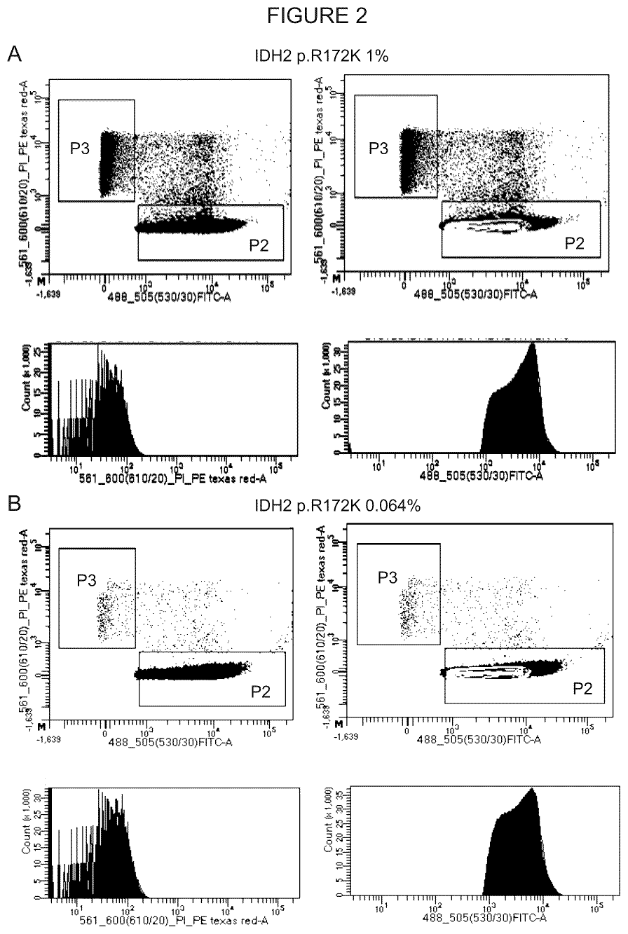

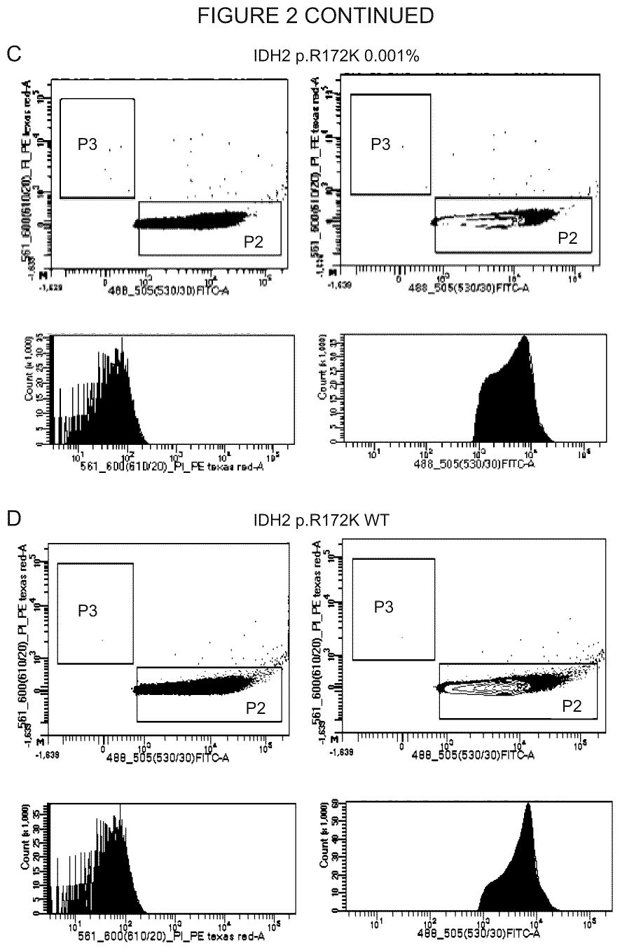

The single tube SuperRCA assay was used to analyse samples with varying levels of the IDH2 p.R172K mutation spiked into the sample, ranging from 1% to 0% (comprising a wildtype sample). The SuperRCA products were detected using BD Fortessa flow cytometer (FIG. 2). The number of events detected by flow cytometry for each sample with varying abundances of IDH2 p.R172K mutation are displayed in Tables 1 to 4 below. P3 is the probe detecting mutant events, and P2 is the probe detecting wildtype events. As can be seen from the flow diagrams in FIG. 2 and the data in Tables 1 to 4, the signal from P3 decreases as the level of spike-in mutations in the samples decrease from 1% to 0%, and even at a very low level of mutation of 0.001%, the SuperRCA assay was still able to detect the mutant signal (FIG. 2C), demonstrating the sensitivity of the assay.

Tables 1-4 present the raw data from the flow cytometry readouts revealing, respectively, 1%, 0.064%, 0.001% and 0% (WT) of the IDH2 R172K mutation.

TABLE 1
(FIG. 2A)
IDH2 p.R172K 1%

| Population | #Events | % Parent | Mean | Mean |
|---|---|---|---|---|
| All Events | 1,326,746 | #### | 4,811 | 59 |
| P2 | 1,317,274 | 99.3 | 4,827 | 22 |
| P3 | 6,502 | 0.5 | 62 | 5,652 |

TABLE 2
(FIG. 2B)
IDH2 p.R172K 0.064%

| Population | #Events | % Parent | Mean | Mean |
|---|---|---|---|---|
| All Events | 1,675,508 | #### | 4,289 | 32 |
| P2 | 1,672,568 | 99.8 | 4,291 | 29 |
| P3 | 393 | 0.0 | 55 | 5,315 |

TABLE 3
(FIG. 2C)
IDH2 p.R172K 0.001%

| Population | #Events | % Parent | Mean | Mean |
|---|---|---|---|---|
| All Events | 1,668,478 | #### | 5,086 | 35 |
| P2 | 1,667,261 | 99.9 | 5,083 | 35 |
| P3 | 9 | 0.0 | 136 | 4,300 |

TABLE 4
(FIG. 2D)
IDH2 p.R172K WT (0%)

| Population | #Events | % Parent | Mean | Mean |
|---|---|---|---|---|
| All Events | 1,774,964 | #### | 5,530 | 36 |
| P2 | 1,774,369 | 100.0 | 5,521 | 35 |
| P3 | 1 | 0.0 | −42 | 2,041 |

### Example 2: Detection of the Ultralow Frequency AML Point Mutation IDH2 p.R172K Using SuperRCA or ddPCR Assays

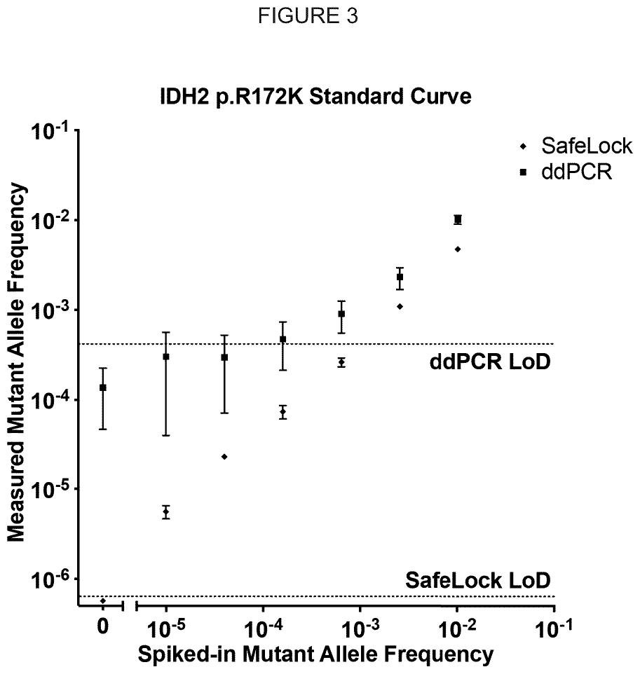

The PCR amplicons targeting DNA sequences containing IDH2 p.R172K mutations were applied in the spike-in DNA samples for the SuperRCA pre-amplification step. Then the SuperRCA protocol was applied on the amplified PCR products to estimate the mutant allele frequency in each spike-in sample, and the SuperRCA products were analyzed with Cytoflex flowcytometers. For the corresponding ddPCR assay, the same spike-in DNA samples was used in the Bio-Rad ddPCR platform with 66 ng per replicate and performed in duplicates. The results as shown in FIG. 3 demonstrate that the SuperRCA assay has a lower limit of detection (LoD) and successfully detected mutant DNA in up to 100,000-fold excesses of normal DNA in comparison to ddPCR, which revealed substantially greater variability and insufficient sensitivity to detect very low frequency mutations.

### Example 3: Monitoring Disease Progression in an AML Patient with the IDH2 p.R140Q Mutation Using SuperRCA, NGS and ddPCR Assays

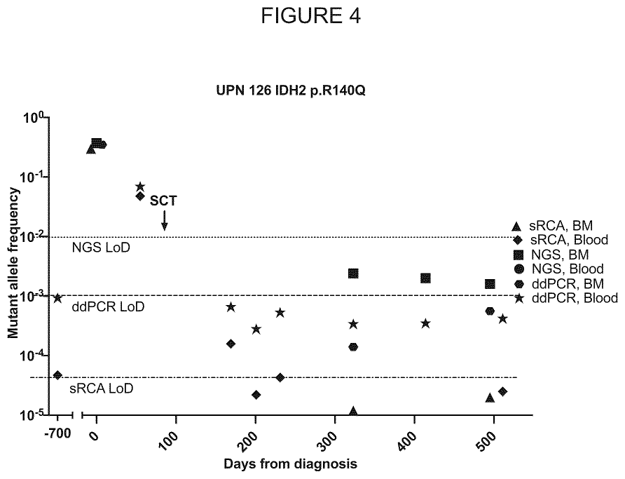

Samples from an AML patient (UPN126) with the IDH2 p.R140Q mutation were analyzed at several time points using SuperRCA, NGS and ddPCR assays to explore the suitability of SuperRCA probing for monitoring the course of disease (FIG. 4). Patient UPN126 was previously diagnosed with primary myelofibrosis associated with a JAK2-mutation, which had transformed to a secondary AML two years after the initial diagnosis. NGS at AML diagnosis, carried out by the clinical collaborators at the Uppsala University Hospital with Trusight NGS panel and sequenced with Illumina MiSeq platform, revealed the presence of an IDH2 p.R140Q mutation alongside the previously known JAK2-mutation. Retrospective analysis of the initial bone marrow sample in the myelofibrosis stage was negative for the IDH2 p.R140Q mutation when analyzed by both SuperRCA and ddPCR assays, indicating a clonal evolution upon AML transformation. The PCR amplicons targeting DNA sequences containing IDH2 p.R140Q mutations were applied in the patient DNA samples for the SuperRCA pre-amplification step. Then the SuperRCA protocol was applied on the amplified PCR products to estimate the mutant allele frequency in each time point sample, and the SuperRCA products were analyzed with Cytoflex flow cytometer. For the corresponding ddPCR assay, the same DNA samples was used in the Bio-Rad ddPCR platform with 66 ng per replicates and performed in duplicates using IDH2 p.R140Q ddPCR assay pre-validated by Bio-Rad. The patient achieved a CR after the first course of intensive chemotherapy but remained MRD-positive at approximately 2% (measured by flow cytometry of bone marrow cells with CD34, CD117, CD13 and DR⁺ markers) after 3 courses of intensive chemotherapy. An allogeneic SCT was performed on day 139. The patient was subsequently monitored by analyzing consecutive blood and BM samples through ddPCR and NGS and all six samples collected post-SCT revealed undetectable levels of the IDH2 p.R140Q mutation by both assays, indicating that this patient has remained in remission with a follow up of more than two years post SCT. The blood sample collected 3 months post SCT was positive by SuperRCA but under the detection limit for ddPCR (FIG. 4), demonstrating the higher level of sensitivity of the SuperRCA assay.

1. A method for detecting a target nucleic sequence in a target nucleic acid molecule, said method comprising:
(a) providing a reaction mixture comprising amplicons of the target nucleic acid sequence, the amplification polymerase, and an excess of amplification primers relative to the amplicons, wherein the amplicon is a single- or double-stranded linear molecule and comprises a single copy of the target nucleic acid sequence;
(b) reducing the excess of primers from the reaction mixture of (a);
(c) if necessary denaturing double-stranded amplicons into single strands;
(d) contacting the reaction mixture with a ligation mix comprising a ligase enzyme and performing a ligation reaction to ligate the 5′ and 3′ ends of the amplicons to circularise the amplicons, wherein the ligation reaction is performed under conditions in which the ability of the amplification polymerase to extend the hybridised 3′ end of the amplicon on the ligation template is inhibited;
(e) adding to the reaction mixture from (d) an RCA mix comprising one or more RCA reagents including at least a RCA polymerase and performing a first RCA reaction using the circularised amplicons as a first RCA template to generate a first RCA product (RCP) comprising multiple repeats of a complementary copy of the target nucleic acid sequence in the amplicon;
(f) heating to inactivate the RCA polymerase;
(g) contacting the first RCP with padlock probes specific for the target nucleic acid sequence and allowing the padlock probes to hybridise to the complementary copies in the multiple repeats;
h) directly or indirectly ligating the hybridised padlock probes to circularise the hybridised padlock probes, wherein the ligation reaction is performed under conditions in which the ability of polymerase to extend the hybridised 3′ end of the padlock probe is inhibited;
(i) removing or rendering inert unligated padlock probes in the reaction mixture of (h);
(j) performing a second RCA reaction using the circularised padlock probes as second RCA templates to generate second RCPs containing multiple repeat complementary copies of the circularised padlock probes;
wherein steps (a) to (j) are performed in a single reaction vessel and temperature is controlled during the steps; and wherein steps (g) to (j) are optionally repeated one or more times;
(k) detecting the second or final RCPs to detect the circularised padlock probes, and thereby the target nucleic sequence.

2. The method of claim 1, wherein in step (b) the excess of primers is reduced by enzymatic digestion and/or by dilution of the reaction mixture.

3. The method of claim 1 or claim 2, wherein the method comprises a preceding step of performing an amplification reaction to generate amplicons of the target nucleic acid sequence.

4. The method of any one of claims 1 to 3, wherein the amplicons are or have been generated by PCR.

5. The method of any one of claims 1 to 4, wherein the method is for detecting a variant target nucleic acid sequence in a target nucleic acid molecule in a sample, and steps (g) to (k) comprise:
(g) contacting the first RCP with two or more padlock probes each comprising target binding regions specific for different variants of the target nucleic acid sequence and allowing the probes to hybridise to their target sequence complements in the multiple repeats;
(h) directly or indirectly ligating the padlock probes which have hybridised to their variant target sequence complements, to circularise the hybridised padlock probes;
(i) removing or rendering inert unligated padlock probes in the reaction mixture of (h);
(j) performing second RCA reactions using the circularised padlock probes as second RCA templates to generate second RCPs containing multiple repeat complementary copies of the circularised padlock probes;
(k) detecting the second or final RCPs to identify the circularised padlock probes, and thereby the variant target nucleic acid sequence.

6. The method of any one of claims 1 to 5, wherein the method is performed in multiplex to detect multiple different target nucleic acid sequences in one or more target nucleic acid molecules.

7. The method of any one of claims 1 to 6, wherein step (b) further comprises enzymatically digesting the amplification polymerase.

8. The method of any one of claims 2 to 7, wherein the enzymatic digestion of the primers is performed by contacting the reaction mixture with exonuclease, or where the primers contain uracil residues, with uracil DNA glycosylase (UDG).

9. The method of any one of claims 2 to 8, wherein after said enzymatic digestion, the digestion enzyme is inactivated by enzymatic digestion or by heating.

10. The method of any one of claims 7 to 9, wherein proteinase K is added to degrade the amplification polymerase, and exonuclease where it is used, and wherein the method further comprises heating to inactivate said proteinase K.

11. The method of any one of claims 1 to 4 and 6 to 10, wherein step (b) comprises adding a volume of diluent to the reaction mixture to dilute it at least 200×.

12. The method of any one of claims 1 to 11, wherein in step (d) the reaction mixture is also contacted with a ligation template.

13. The method of claim 12, wherein in step (d) a two-step hybridisation of the ligation template is performed, to anneal the 5′ end of the amplicon to the ligation template at a first temperature, and then reducing the temperature to allow the 3′ end of the amplicon to anneal to the ligation template.

14. The method of any one of claims 1 to 13, wherein:
(i) in step (e), the RCA mix comprises the RCA polymerase and dNTPs; and/or
(ii) in step (e) the first RCA is primed by the ligation template; and/or
(iii) the ligase enzyme in step (d) is thermophilic.

15. The method of claim 14, wherein in part (iii) said denaturation step (c) takes place concurrently with or after adding the ligation mix in step (d), and then the temperature is reduced for the ligation reaction; and/or wherein the ligase from step (d) performs the ligation in step (h).

16. The method of any one of claims 1 to 15, wherein in step (g) a two-step hybridisation of the padlock probe is performed, to anneal the 5′ end of the padlock probe to the first RCP at a first temperature, and then reducing the temperature to allow the 3′ end of the padlock probe to anneal to the first RCP.

17. The method of any one of claims 1 to 15, wherein in step (i):
(a) unligated padlock probes are removed by enzymatic digestion with exonuclease, and said method further comprises heating to inactivate the exonuclease prior to the second RCA of step (j); or
(b) unligated padlock probes are removed by enzymatic digestion by an RCA polymerase with 3′ exonuclease activity before the second RCA reaction is initiated.

18. The method of any one of claims 1 to 4 and 6 to 17, wherein step (i) comprises adding a volume of diluent to the reaction mixture to dilute it at least 4×.

19. The method of any one of claims 1 to 18, wherein the padlock probes are hairpin-forming padlock probes comprising a hairpin loop structure which comprises the 3′ end of the padlock probe, and in step (i) the step of rendering the unligated and unhybridised padlock probes inert comprises extending the 3′end in the hairpin loop structure.

20. The method of any one of claims 1 to 19, wherein detection reagents for detecting the second RCP are included with one or more RCA reagents added to the reaction mixture, or wherein said detection reagents are added after the second RCP has been generated.

21. The method of any one of claims 1 to 20, wherein detection oligonucleotides are hybridised to the second RCP, and wherein in step (k) the second RCP is detected by detecting the detection oligonucleotides.

22. The method of claim 21, wherein the detection oligonucleotides are labelled with detectable labels.

23. The method of any one of claims 1 to 22, wherein the second RCPs are detected by microscopy or by flow cytometry.

24. The method of any one of claims 1 to 23, wherein reagents added to the reaction mixture of step (a) are provided in the same buffer.

25. A kit for use in detecting a target nucleic acid sequence in a target nucleic acid molecule, said kit comprising:
(i) one or more ligation templates which comprises amplicon-binding regions which are capable of hybridising to complementary binding sites in amplicons of the target nucleic acid sequence, allowing the 3′ and 5′ ends of the amplicon to be ligated;
(ii) one or more padlock probes which comprises target-binding regions which are specific for a target nucleic acid sequence; and optionally one of more of:
(iii) an exonuclease;
(iv) proteinase K;
(v) a dilution buffer;
(vi) a ligase;
(vii) a RCA polymerase;
(viii) a RCA primer;
(ix) dNTPs;
(x) detection oligonucleotides capable of hybridising to an RCP generated using a circularised padlock probe as a RCA template.
